SEQUENCE LISTING (5 sequences)

SEQ ID NO: 1 - protein, 6 aa, synthetic construct
GRGDSP

SEQ ID NO: 2 - skipped

SEQ ID NO: 3 - protein, 4 aa, synthetic construct
REDV

SEQ ID NO: 4 - protein, 4 aa, synthetic construct
VAPG

SEQ ID NO: 5 - skipped

(19) United States
(12) Patent Application Publication
(10) Pub. No.: US 2024/0374648 A1
(43) Pub. Date: Nov. 14, 2024

(54) NOVEL THERAPY

(71) Applicant: KING'S COLLEGE HOSPITAL NHS FOUNDATION TRUST, London, Greater London (GB)

(72) Inventors: Anil DHAWAN, London (GB); Celine FILIPPI, London (GB)

(21) Appl. No.: 18/686,792

(22) PCT Filed: Sep. 27, 2022
(86) PCT No.: PCT/EP2022/076774

§ 371 Date: Feb. 26, 2024

(30) Foreign Application Priority Data
Sep. 27, 2021 (GB) 2113791.4

(51) Int. Cl. A61K 35/28; A61K 9/50; A61K 35/407; A61K 47/20; A61K 47/26; A61K 47/62; A61P 1/16

(52) U.S. Cl. CPC A61K 35/28; A61K 9/5036; A61K 9/5089; A61K 35/407; A61K 47/20; A61K 47/26; A61K 47/62; A61P 1/16

(57) ABSTRACT

This invention relates to a composition useful in the treatment of liver disease, to methods for preparing the composition and its use in therapy. The composition comprises a combination of hepatocytes and mesenchymal stromal cells (MSCs), wherein said cells are co-encapsulated within alginate microbeads derivatised with a peptide comprising an RGD motif.

### TECHNICAL FIELD

This invention relates to a novel pharmaceutical composition useful in the treatment of liver disease, to methods for preparing the composition and its use in therapy.

### BACKGROUND

Acute liver failure (ALF) is a rare medical emergency that carries a high mortality without liver transplantation. However, liver transplantation depends on the timely availability of a suitable donor organ and whole organ replacement requires life-long immunosuppression and is linked to a number of complications. The liver has tremendous regenerative potential: if the patient's failing liver could be supported until regeneration has happened, whole organ replacement and its associated complications could be avoided.

It has been demonstrated that partial replacement of a patient's liver with a healthy donor liver could lead to native liver regeneration, thus making the transplanted liver redundant, in up to 70% of the patients. Thus, a small mass of liver tissue may be sufficient to support the patient and a transitory procedure can replace life-long whole liver replacement. Transplantation of hepatocytes (liver cells rather than an organ) has been shown to improve synthetic and detoxification functions in small animal models with subsequent human application in patients with ALF (Dhawan et al., 2010, Nature Reviews Gastroenterology & Hepatology, 7(5), 288-298; Baccarani et al., 2005, Transplantation Proceedings, Vol. 37 (6), pp. 2702-2704). The advantages of hepatocyte transplantation in this context are considerable. For example, (i) hepatocytes may be derived from livers which are unsuitable for transplantation; (ii) isolated cells can be kept frozen for years and used off-the-shelf, something that is impossible with entire organs, thereby eliminating the wait for an appropriate organ; (iii) cells isolated from one liver could treat more than one patient, thereby reducing the need for donor organs; and (iv) this provides options to infants and small children for whom the wait for an appropriately sized organ, most often from a size matched donor, may be extremely prolonged. Earlier clinical experiences with human hepatocytes in ALF have been only partly successful when cells were injected either in the liver, or the peritoneal cavity, mainly because of rejection and use of immunosuppression in very sick patients, which increases the risk of infection.

The technique of hepatocyte transplantation within alginate microbeads infused into the peritoneal cavity is much less invasive than liver transplantation and, as the alginate coating protects the cells against the body's immune system, avoids the need for immunosuppression and the major risks which accompany this (Umehara et al., 2001, Surgery, Vol. 130(3), pp. 513-520).

It is known that mesenchymal stromal cells (MSCs) improve the survival of hepatocytes and their liver-specific functions in standard cell culture conditions. However, the applicants have found that the beneficial effects of MSCs on primary human hepatocytes may be lost when the cells were encapsulated in a standard alginate (unpublished data).

Co-encapsulation of hepatocytes with bone marrow mesenchymal stem cells (MSCs) was reported to improve hepatocyte-specific functions in vitro and in vivo (Xiao-Lei Shi et al., 2009, Transplantation, 88(10): 1178-1185). In that instance, the cells were encapsulated in an alginate-poly-L-lysine-alginate composite which had been treated with sodium citrate to depolymerise the core.

The use of a wide range of cell adhesion peptides is known to enhance the viability and functionality of adherence dependent cells under certain circumstances. However, the enhancement is not achieved in all circumstances. For instance, Follin et al., 2015 (Cytotherapy, 17(8), pp. 1104-1118), reports that the RGD peptide did not impact on the efficacy of alginate hydrogels containing ASCs when used in the treatment of heart disease.

Furthermore, it has also been reported that the hydrogels including cell adhesion peptides may enhance or induce cell proliferation (T. Andersen et al., 2015, Microarrays, 4: 133-161), a feature which could be problematic in relation to encapsulated cells, as this would cause rupture of the capsules and release of cells.

The applicants have found, however, that any effects are highly dependent upon the nature of the composition and can be enhanced, in particular, by the use of specifically modified alginates in the encapsulation process.

### SUMMARY OF THE INVENTION

The present invention provides a composition comprising a combination of hepatocytes and mesenchymal stromal cells (MSCs), wherein said cells are co-encapsulated within alginate microbeads, characterised in that the alginate is derivatised with a peptide comprising an Arg-Gly-Asp (RGD) motif.

The invention also provides a method for the treatment of liver disease, comprising administering the compositions of the invention to a subject in need thereof. Also provided is the use of the compositions of the invention in the treatment of liver disease.

Also provided is a method for preparing a composition as described above, said method comprising obtaining hepatocytes and MSCs and admixing the hepatocytes and MSCs with a solution of an alginate which has been derivatised with a peptide comprising an RGD motif, and cross-linking the alginate to produce microbeads in which the hepatocytes and MSCs are co-encapsulated.

The present invention also provides a pharmaceutical composition comprising a therapeutically effective amount of the composition of the invention.

Kits comprising components for performing the invention are also provided. The kit according to the invention comprises any one or more of: MSCs, hepatocytes, alginate derivatised with a peptide comprising an Arg-Gly-Asp (RGD) motif, and optionally a suitable transplant, storage or cryopreservation media.

A further aspect of the invention provides use of the composition or pharmaceutical composition of the invention in the treatment or prophylaxis of liver disease. The use comprises administering to a patient in need thereof an effective amount of a composition as described above.

Also provided is the use of alginate derivatised with a peptide comprising an Arg-Gly-Asp (RGD) motif for the co-encapsulation of hepatocytes and MSC.

Also provided is the use of alginate derivatised with a peptide comprising an Arg-Gly-Asp (RGD) motif to potentiate the effects of MSC on hepatocytes.

### DETAILED DESCRIPTION

The applicants have found that the introduction specifically of an RGD motif into an alginate used for the co-encapsulation of hepatocytes and mesenchymal stromal cells (MSCs) enhanced results, particularly in terms of long-term viability and function of the encapsulated cells. Other adhesion peptides known in the art did not produce beneficial results under these circumstances, as described further below. Thus, the finding that the inclusion of an RGD peptide did not disrupt the microbead structure but enhanced efficacy was surprising, particularly in the light of references such as Andersen et al. and Follin et al. (supra.).

A first aspect of the present invention provides a composition comprising a combination of hepatocytes and mesenchymal stromal cells (MSCs), wherein said cells are co-encapsulated within alginate to form microbeads, characterised in that the alginate is derivatised with a peptide comprising an Arg-Gly-Asp (RGD) motif.

The compositions of the invention advantageously lead to improved hepatic functions relative to controls. The improved hepatic functions may be manifested through any one or more of: i) increased cell viability; ii) biosynthetic capacity, for example as assessed by measurement of proteins typically produced by hepatocytes, such as increased albumin and/or al-antitrypsin (AAT) production; iii) metabolic capacity, for example, as assessed by measurement of the hepatocyte's capacity to detoxify ammonia through the urea cycle; iv) detoxifying capacity, for example, as assessed by increased phase 1 and/or 2 metabolic activity.

Further advantageously, the compositions of the invention, in use (i.e., in vivo) and/or upon retrieval, remain intact, retain their original shape, size and appearance, and are substantially clear of any coating. The compositions of the invention, in use and/or upon retrieval, also advantageously do not show any one or more of the following signs: substantial shrinkage, damage, absorption, metabolism, excretion, distribution outside of the peritoneum. In contrast, the inventors found controls to show partial dissolving and lower levels of retrieval, with evidence of shrinkage and damage, suggesting absorption, excretion and distribution of the control microbeads outside of the peritoneum. Some control microbeads, when retrieved, were also partially or fully coated in what was presumed to be the host's immune cells.

Further advantageously, performing the invention showed no signs of distress in pre-clinical subjects, who gained weight as normal and showed results similar to controls in terms of full blood count, coagulation, electrolytes, liver and renal function.

It was surprisingly found that the specific use of an RGD derivatised alginate appears to potentiate the effects of MSCs on hepatocytes, whilst other ECM-mimicking peptides had no effects. Albumin, and to a greater extent human al-antitrypsin production levels were found by the inventors to be increased compared to controls. For example, the addition of MSC to the MVG GRGDSP-encapsulated hepatocytes gave a 12-fold increase in AAT secretion at day 3, compared to a 4-fold increase when using control microbeads (SLG20: non-RGD microbeads). At day 7, the secretion of AAT was undetectable in cells encapsulated in pure alginate (SLG20), whilst still steady in hepatocytes in MVG GRGDSP alginate and further increased with MSC.

The controls are suitably MSC and hepatocytes co-encapsulated in an alginate that does not comprise the RGD peptide modification. A person skilled in the art would readily be aware of other suitable controls.

### RGD (Arg-Gly-Asp) Motif

The RGD motif may be part of a longer peptide, in particular a peptide of up to 10 amino acids, for example up to 6 amino acids, such as a peptide having the sequence GRGDSP (SEQ ID NO: 1). This is a highly conserved peptide present in a number of extracellular matrix proteins, and which favour cell adhesion. The RGD motif may suitably be comprised in a peptide of up to 9, 8, 7, 6, 5 or 4 peptides in length, or may simply comprise the three amino acids R-G-D (SEQ ID NO: 2).

Methods of making peptides are well known to those of skill in the art. For example, peptides can be produced recombinantly (see Sambrook et al., Molecular Cloning: A Laboratory Manual (3 ed.), Cold Spring Harbor Laboratory Press, Cold Spring Harbor, N.Y., USA (2001)) or synthesised.

The peptide comprising an RGD motif may be derived from EGF, VEGF, b-FGF, FGF, TGF, TGF-β, proteoglycans or fragments or derivatives thereof.

The peptide sequence is suitably chemically coupled or otherwise linked to the alginate. Methods for coupling peptides to polymer backbones are well known in the art. The peptide sequence may be linked to a low, medium or high molecular weight alginate. Suitable alginates derivatised with a peptide comprising an RGD motif are also commercially available, such as MVG GRGDSP available from NOVATACH™ (cat. #4270129).

### Alginates

Alginates are salts of alginic acid. Alginic acid is a polyuronic acid made of two uronic acids: D-mannuronic acid and L-guluronic acid. Alginates have the chemical formula (C₆H₈O₆)n and occur naturally in various algae (such as Laminaria japonica), seaweed and bacteria. Alginates can also be synthetic or semisynthetic. The ratio of mannuronic acid and guluronic acid varies with factors such as seaweed species, plant age, and part of the seaweed (e.g., stem, leaf, etc.). Alginic acid is substantially insoluble in water. It forms water-soluble salts with alkali metals. Alginates are hydrophilic colloids. The properties of alginates are described further in Gaserod et al., 1998, Biomaterials 19(20): 1815-25.

Alginates can be characterised by the ratio of mannuronic acid (M) and guluronic acid (G). Increasing the proportion of G units results in a more viscous material. Viscosity of an aqueous solution of alginates is also influenced by the concentration of the alginate and the chain length (i.e., degree of polymerisation), and thus the molecular weight, of the individual alginate molecules. Viscosity is also influenced, for example, by the shear rate of the solution, temperature, and acid conditions. Alginate molecules are categorised as either low viscosity alginates (LV alginates), medium viscosity alginates (MV alginates) or high viscosity alginates (HV alginates). Additional categories such as ultra-high viscosity (e.g. UHV alginates) or very low viscosity alginates (e.g. VLV alginates) are sometimes used to describe certain alginate molecules. Alginates are typically referred to as “M” alginates when they have at least 50% mannuronic acid and no more than 50% guluronic acid. Alginates are typically referred to as “G” alginates when they have at least 60% guluronic acid and no more than 40% mannuronic acid.

The viscosity and subunit descriptors may be combined to categorise the alginates. An MVG alginate is a medium viscosity alginate having at least 60% guluronic acid; an MVM alginate is a medium viscosity alginate having a least 50% mannuronic acid; a HVM alginate is a high viscosity alginate having at least 50% mannuronic acid; a HVG alginate is a high viscosity alginate having at least 60% guluronic acid; a LVM alginate is a low viscosity alginate having at least 50% mannuronic acid and a LVG alginate is a low viscosity alginate having at least 60% guluronic acid.

The alginate for use in the invention may be a G alginate, an M alginate, an MVG alginate, an MVM alginate or an LVM alginate.

Alginates of various forms are commercially available, for example, under the PRONOVA® and NOVATACH™ names from NovaMatrix® (https://novamatrix.biz). A suitable alginate for use in the invention is NOVATACH™ MVG GRGDSP (cat. #4270129), although any “RGD” alginate may be suitable for use in the invention.

Commercially available forms of alginates are available having a molecular weight in the range of 9,000-600,000 g/mol. The viscosity of an alginate solution can be manipulated by changing the concentration of the alginate or by using materials with varying chain length (i.e., molecular weight). In a particular embodiment, the alginate used in the composition is an alginate having a high molecular weight, for example, in excess of 75,000 g/mol, such as in excess of 100,000 g/mol; 200,000 g/mol; 300,000 g/mol; 400,000 g/mol; 500,000 g/mol; or in excess of 600,000 g/mol. A low molecular weight alginate is an alginate having a molecular weight of less than 75,000 g/mol, such as less than 60,000 g/mol; 50,000 g/mol; 40,000 g/mol; 30,000 g/mol; 20,000 g/mol; or less than 10,000 g/mol. The molecular weight of an alginate can be determined by any method known in the art. A suitable alginate for use in the invention is a high molecular weight alginate, such as NOVATACH™ MVG GRGDSP (cat. #4270129). Other high or low molecular weight alginates may also be suitable for use in the invention, provided that the alginate is derivatised with a peptide comprising an RGD motif.

In a particular embodiment, the alginate has a high guluronic acid (G) content. In particular, the ratio of guluronic acid (G) to mannuronic acid (M) (G/M ratio) is at least 1.5:1. A particular example of such an alginate is available from NovaMatrix® as NOVATACH™ MVG GRGDSP. MVG GRGDSP, having a molecular weight higher than 200 kDa, is an example of a high molecular weight alginate.

LVM GRGDSP, having a molecular weight of 75-200 kDa contains the same GRGDSP motif but is an example of a medium molecular weight alginate. G VAPG, and M-REDV are alginates combined with different types of peptides, all meant to allow cell anchoring within the alginate gel. See Table 2 in the Examples section hereinbelow for further information on alginates useful in the invention.

In a particular embodiment, the alginate is an alkali or alkaline earth metal salt, such as a potassium or sodium alginate, and in particular sodium alginate.

### Mesenchymal Stromal Cells (MSCs)

Mesenchymal stromal cells, also referred to as mesenchymal stem cells, are multipotent stem cells that can be differentiated into a variety of cell types including osteoblast, chondrocytes (cartilage cells), myocytes (muscle cells), adipocyte (fat cells), etc.

MSCs suitable for use in the invention may be derived from various sources including neonatal tissues such as umbilical cords or placenta, as well as from adult tissue including bone marrow, adipose tissue and peripheral blood. In a particular embodiment, the MSCs are derived from umbilical cords. Methods for isolating and identifying mesenchymal stem cells are well known in the art.

MSCs may be identified by one or more of the characteristics shown in the Table 1 below reproduced from Horwitz et al., 2006 (Current Opin Hematol, 13(6), pp. 419-425) as proposed by the ISCT Mesenchymal and Tissue Stem Cell Committee.

TABLE 1
Criteria for Identifying Mesenchymal stromal cells (MSCs)

| | | |
|---|---|---|
| 1. Adherence to plastic in standard culture conditions | | |
| 2. Phenotype | Positive (≥95%+) | Negative (<2%+) |
| | CD105 | CD45 |
| | CD73 | CD34 |
| | CD90 | CD14 or CD11b |
| | | CD79a or CD19 |
| | | HLA-DR |
| 3. Differentiation: (by staining of in vitro cell culture) | Bone, Adipose, Cartilage | |

The MSCs may be isolated multipotent stem cells or derived from pluripotent stem cells (e.g., isolated from a subject as multipotent, or derived from embryonic or induced pluripotent stem cell (iPSC)). The term iPSC refers to pluripotent cells derived from differentiated cells. For example, iPSCs may be obtained by overexpression of transcription factors such as Oct4, Sox2, c-Myc and Klf4.

The concentration of MSCs in the composition may be in the range of from about 0.4×10⁶ cells per ml of alginate to about 1.2×10⁶ cells per ml of alginate, optionally from about 0.6×10⁶ cells per ml of alginate to about 1.0×10⁶ cells per ml of alginate or from about 0.7×10⁶ cells per ml of alginate to about 0.9×10⁶ cells per ml of alginate. According to a preferred embodiment, the concentration of MSCs is about 0.8×10⁶ cells per ml of alginate.

### Hepatocytes

The term “hepatocyte” as referred to herein means liver cells, including hepatocyte-like cells. The hepatocytes, including hepatocyte-like cells, may be of human or animal origin, for example porcine in origin. The hepatocytes may be primary hepatocytes, meaning that they are directly isolated from liver tissue or have not undergone significant passaging or subculture in vitro post isolation from a donor subject.

The hepatocytes may be stem cell derived. For example, hepatocytes may be derived from embryonic stem cells (ESCs), human pluripotent stem cells (hPSCs), induced pluripotent stem cells (iPSCs), mesenchymal stem cells, or hepatic progenitor cells.

The hepatocytes may be obtained from a paediatric subject or from an adult subject. The hepatocytes may be obtained from the subject to be treated (autologous) or obtained from an unconnected subject.

Hepatocytes have an important detoxifying role in the organism, mainly mediated by phase 1 and phase 2 enzymes. Phase 1 metabolism results in chemical changes that make a compound more hydrophilic, so it can be effectively eliminated by the kidneys. These reactions usually involve hydrolysis, oxidation or reduction mechanisms and happen in the hepatocytes. The enzymes responsible for most phase 1 reactions belong to a large family of inducible enzymes, called cytochrome P450. The P450 superfamily is divided into families (e.g. CYP1, CYP2, CYP3, etc.) and in subfamilies (labelled with letters A, B, C, etc.). In man, more than 50 isoforms have been isolated with members of the CYP450 families; 1, 2 and 3 being the main ones responsible for the metabolism of drugs and other xenobiotics. Among these isoforms, CYP1A1/2 and CYP3A4 are the most studied markers of hepatocyte functions in vitro, as they are responsible of a large number of phase 1 metabolism in humans. The composition of the invention advantageously showed significantly increased CYP1A1/1A2 activity relative to controls.

Phase 2 metabolism takes place if phase 1 is insufficient to clear a compound from circulation, or if it generates a reactive metabolite. These reactions usually involve adding a large polar group (conjugation reaction), such as glucuronide, to further increase the compound's solubility. Many enzymes are involved in the phase 2 metabolism, most of them in the liver. Phase 2 activity may be determined by the measurement of resorufin metabolism, with a decrease in resorufin being indicative of phase 2 metabolism.

The concentration of hepatocyte cells in the composition of the invention may be in the range of from about 0.5×10⁶ cells per ml of alginate to about 5.0×10⁶ cells per ml of alginate, optionally from about 2.0×10⁶ cells per ml of alginate to about 3.0×10⁶ cells per ml of alginate. According to a preferred embodiment, the concentration of hepatocyte cells is about 2.5×10⁶ cells per ml of alginate.

### Hepatocytes to MSC Ratio

The ratio of hepatocytes to MSCs in the composition may vary, but in a suitable embodiment range from about 1:1 to 5:1. In a particular embodiment, hepatocytes are present in excess, for example, the ratio of hepatocytes to MSCs is in excess of 1.5:1, such as about 2:1 or 3:1. In another embodiment, the ratio of hepatocytes to MSCs in the composition is about 1:1.

### Microbead/Microcapsule

The terms “microbead”, “microcapsule” and the like are used interchangeably herein to describe hepatocytes, optionally together with mesenchymal stromal cells (MSCs), encapsulated in alginate. Microcapsules, in general, have a liquid core.

The microbeads of the invention comprise a combination of hepatocytes and MSCs co-encapsulated within an alginate derivatised with a peptide comprising an RGD motif; however, the control microbeads may differ in terms of the type of alginate used or whether both hepatocytes and MSCs are present.

### Transplant, Storage, Cryopreservation Media

According to a preferred embodiment, the composition of the invention is administered in a transplant medium.

Suitable transplant media would be well known to those skilled in the art. An example of a suitable transplant medium is one comprising or consisting of: CMRL-1066 (Connaught Medical Research Laboratories Medium) without L-Glutamine, without Phenol red, with Sodium carbonate and (3.3%) Human Albumin Solution.

The composition of the invention may comprise up to and including 30 ml of alginate microbeads in up to and including 30 ml of transplant medium per kg body weight of the subject; more particularly up to and including 20 ml of alginate microbeads in up to and including 20 ml of transplant medium per kg body weight of the subject; or up to and including 10 ml of alginate microbeads in up to and including 10 ml of transplant medium, per kg body weight of the subject. The composition of the invention may also be administered in a 2:1 v/v ratio of microbeads:transplant medium. The concentration of hepatocytes and MSCs in the composition is as described herein. Furthermore, the ratio of hepatocytes to MSCs in the composition is as described herein.

If the composition is not for immediate use, it may be stored in a suitable storage or cryopreservation medium. Examples of suitable media may include UW (University of Wisconsin cold storage medium) and DMSO.

### Subject

The subject suitable for treatment as described herein includes mammals, such as a human, non-human primate, cow, horse, pig, sheep, goat, dog, cat, rabbit, or rodent. In preferred embodiments, the subject is a human. Practice of methods described herein in other mammalian subjects, especially mammals that are conventionally used as models for demonstrating therapeutic efficacy in humans (e.g., murine, primate, porcine, canine, or rabbit animals), is also encompassed. Standard dose-response studies may be used to optimise dosage and dosing schedule. The subject may be a paediatric or adult subject.

### Liver Disease

Aspects of the invention as described herein may be used in the treatment of acute or chronic liver disease. For example, in the treatment of Acute Liver Failure (ALF) or in the in the treatment of chronic liver diseases, such as alcohol and chronic viral hepatitis.

In particular, the compositions of the invention are useful in the treatment of liver disease, particularly ALF, and more particularly in paediatric patients.

### Method of Treatment

According to a second aspect of the invention, there is provided a method for the treatment of liver disease, comprising administering the compositions of the invention to a subject in need thereof.

“Administering” refers to the physical introduction of the composition of the invention to a subject using any of the various known methods and systems for delivery. Examples include intraperitoneal delivery or injection into the existing liver. The composition of the invention is preferably administered to the peritoneal cavity of a subject and suitably remains in the peritoneum until such time as the native organ regenerates or a suitable liver is accepted for transplantation. The composition of the invention once administered may remain in the peritoneal cavity of a subject for up to one week, two weeks, three weeks, one month, two months, three months, four months, five months, six, months, seven months, eight months, nine months, ten months, eleven months, twelve months, two years, three years, four years, five years, ten years, fifteen years, twenty years, thirty years or more, or until the desired result is achieved, e.g. until the native organ is sufficiently regenerated.

Administering may be performed, for example, once, a plurality of times, and/or over one or more extended periods. The compositions of the invention are preferably administered to a subject in need as a single dose. Preferably the dose administered is in the range of between about 5 ml to 30 ml microbeads per kg of body weight of the subject, such as a dose in the range of between about 5 ml to 30 ml per kg body weight or 7 ml to 25 ml per kg of weight of the subject and preferably wherein the dose administered is in the range of between about 12 ml to 22 ml per kg of body weight of the subject. The dose administered to the subject may comprise up to about 5 ml, 6 ml, 7 ml, 8 ml, 9 ml, 10 ml, 11 ml, 12 ml, 13 ml, 14 ml, 15 ml, 16 ml, 17 ml, 18 ml, 19 ml, 20 ml, 21 ml, 22 ml, 23 ml, 24 ml, 25 ml or up to about 30 ml or more of microbeads per kg of body weight of the subject. The microbeads may suitably be administered together with a transplant medium as described herein. The ratio of transplant medium to microbeads may be about 1:1. In one embodiment of the invention, about 10 ml of microbeads are administered to a subject in about 10 ml of transplant media, the microbeads comprising a ratio of hepatocytes to MSCs in the range of between about 1:1 to 5:1.

The composition of the invention is suitably fully cross-linked at the time of delivery to a subject.

Advantageously, following transplantation, the compositions of the invention remain substantially intact, retaining their shape and size and original visual appearance. This is contrast with other types of alginates, where partial dissolving, shrinkage and damage was seen, and levels of retrieval were lower, suggesting substantial absorption, excretion and distribution outside of the peritoneum.

### Prophylaxis

The composition of the invention may also be used prophylactically in the treatment of liver disease, by administering the composition to a subject having an existing disease or condition in order to lessen, reduce or improve at least one symptom associated with the disease and/or to slow down, reduce or block the progression of the disease.

The method of treatment may comprise prophylactic use of the composition of the invention. In this respect, the composition may be administered to a subject who has not yet contracted the disease and/or who is not showing any symptoms of the disease to prevent or impair the cause of the disease or to reduce or prevent development of at least one symptom associated with the disease. The subject may have a predisposition for, or be thought to be at risk of developing, the disease. The method of treatment may advantageously bridge patients to full recovery or liver transplant.

Thus, a further aspect of the invention comprises a method for the treatment or prophylaxis of liver disease, said method comprising administering to a patient in need thereof an effective amount of a composition as described above.

### Preparation of Composition

According to a third aspect, the invention provides a method for preparing a composition as described above, said method comprising:
i) obtaining hepatocytes and MSCs; and
ii) admixing the hepatocytes and MSCs with a solution of an alginate which has been derivatised with a peptide comprising an RGD motif; and
iii) cross-linking the alginate to produce microbeads in which the hepatocytes and MSCs are co-encapsulated; and optionally
iv) storing the microbeads in a suitable storage or cryopreservation medium until ready for use; or when ready for clinical use
v) adding the microbeads to a suitable transplant medium.

FIG. 2 shows a schematic representation of an example of a process for the co-encapsulation of hepatocytes and MSCs in alginate.

The ratios and concentrations of hepatocytes and MSC are as defined herein.

The storage, cryopreservation and transplant media are as described herein.

As used herein, a “cross-linking agent” can be any agent which induces the gelation of the alginates. The cross linking is suitably effected using a solution containing any divalent cations (e.g., Ca²⁺, Ba²⁺, Sr²⁺). The amount of cross-linking agent may readily be determined by a person skilled in the art.

### Pharmaceutical Composition

The present invention also provides a pharmaceutical composition comprising a therapeutically effective amount of the composition of the invention. The pharmaceutical composition preferably includes a pharmaceutically acceptable carrier, diluent or excipient (including combinations thereof). Acceptable carriers or diluents for therapeutic use are well known, and are described, for example, in Remington's Pharmaceutical Sciences, Mack Publishing Co., (A. R. Gennaro edit. 1985). The choice of pharmaceutical carrier, excipient or diluent can be selected with regard to the intended route of administration and standard pharmaceutical practice. The pharmaceutical compositions may comprise as—or in addition to—the carrier, excipient or diluent any suitable binder(s), lubricant(s), suspending agent(s), coating agent(s) or solubilising agent(s).

Examples of pharmaceutically acceptable carriers include, for example, water, salt solutions, alcohol, silicone, waxes, petroleum jelly, vegetable oils, polyethylene glycols, propylene glycol, liposomes, sugars, gelatin, lactose, amylose, magnesium stearate, talc, surfactants, silicic acid, viscous paraffin, fatty acid monoglycerides and diglycerides, petroethral fatty acid esters, hydroxymethyl-cellulose, polyvinylpyrrolidone, and the like.

According to a preferred embodiment, the pharmaceutical composition comprises, essentially consists of, or consists of a therapeutically effective amount of the composition of the invention, additionally together with a transplant medium when ready for clinical use.

### Kit

According to a further aspect of the present invention, there is provided a kit comprising components for performing the invention. The kit may therefore comprise any one or more of: MSCs, hepatocytes, alginate derivatised with a peptide comprising an Arg-Gly-Asp (RGD) motif, and optionally a suitable transplant, storage or cryopreservation media.

The kit may further comprise directions for the appropriate preparation and/or use of the components therein. The kit may further comprise tools useful for the preparation and/or delivery of the microbeads to a subject, as described herein.

### Use

A further aspect of the invention provides the composition or a pharmaceutical composition according to the invention for use in the treatment or prophylaxis of liver disease. The use comprises administering to a patient in need thereof an effective amount of a composition as described above.

Also provided is the use of alginate derivatised with a peptide comprising an Arg-Gly-Asp (RGD) motif for the co-encapsulation of hepatocytes and MSC.

Also provided is the use of alginate derivatised with a peptide comprising an Arg-Gly-Asp (RGD) motif to potentiate the effects of MSC on hepatocytes.

Throughout the description and claims of this specification, the words “comprise” and “contain” and variations of the words, for example “comprising” and “comprises”, mean “including but not limited to”, and do not exclude other components, integers or steps. Moreover, the singular encompasses the plural unless the context otherwise requires: in particular, where the indefinite article is used, the specification is to be understood as contemplating plurality as well as singularity, unless the context requires otherwise.

Preferred features of each aspect of the invention may be as described in connection with any of the other aspects. Within the scope of this application, it is expressly intended that the various aspects, embodiments, examples and alternatives set out in the preceding paragraphs, in the claims and/or in the following description and drawings, and in particular the individual features thereof, may be taken independently or in any combination. That is, all embodiments and/or features of any embodiment can be combined in any way and/or combination, unless such features are incompatible.

### BRIEF DESCRIPTION OF THE DRAWINGS

One or more embodiments of the invention will now be described, by way of example only, with reference to the accompanying drawings, in which:

FIG. 1 is a schematic representation of the hepatocyte-MSC-peptide alginate microbead. Hepatocytes (dark colour) are co-encapsulated with MSCs (light colour) within a peptide-alginate gel microbead. The gel pores are wide enough to allow the substrates of metabolism to reach the cells (including oxygen) and the waste products to be secreted out of the bead. The pores are narrow enough to prevent any access to the encapsulated cells by immune cells.

FIG. 2 is a schematic representation of the process used to prepare hepatocyte/MSC microbeads.

FIG. 3: In vitro viability of cells encapsulated in HMB002 (HMB002 microbeads comprise primary human hepatocytes (HC) and mesenchymal stromal cells (MSCs) encapsulated in MVG GRGDSP alginate). Calcein-AM assay on SLG20 and MVG GRGDSP microbeads containing HC alone or in combination with MSC. Results are shown as mean±SEM. N=3. (B, C) HC microbeads in SLG20 (B) and MVG GRGDSP alginate (C) stained with FDA (fluorescein diacetate, green, viable cells) and PI (propidium iodide, red, dead cells) 9 days after production. Live cells are bright green (FDA), while dead cells are red (PI). Magnification 5×.

FIG. 4: Hepatic functions of HMB002, tested in vitro. (A) Albumin, (B) α1-antitrypsin and (C) urea produced by HC encapsulated in SLG20 and MVG GRGDSP alginate microbeads alone or in combination with MSC. Results are shown as mean±SEM. * p<0.05, ** p<0.01, *** p<0.001, N=3. OMP: omeprazole.

FIG. 5: Phase 1 and 2 metabolism of HMB002. Cytochrome P450 1A1/1A2 (A) and 3A4 (B) activity in alginate microbeads containing hepatocytes with or without MSC after induction with omeprazole and rifampicin, respectively. (C) Phase 2 metabolism analysis through measurement of resorufin conjugation. Results are shown as mean±SEM.** p<0.01, N=3.

FIG. 6: HMB002 retrieval after in vivo transplantation. Alginate microbeads were retrieved 28 days after intraperitoneal transplantation in Sprague Dawley rats. While MVG GRGDSP microbeads (A) could be clearly seen in the intraperitoneal cavity (highly reflective material, white arrow), LVM GRGDSP microbeads (B) were undetectable and a white thin layer, presumably dissolved LVM GRGDSP alginate, was covering the liver (red arrow). The analysis of the retrieved microbeads volume (C) shows a significant lower value for LVM GRGDSP compared to MVG GRGDSP alginate microbeads. Results are shown as mean±SEM, * p<0.05, n=3.

FIG. 7: Human albumin detected in rat plasma after HMB002 transplantation. Animals were transplanted with alginate microbeads containing HC with or without MSC in SLG20 or MVG GRGDSP alginate. Results are shown as mean±SEM. * p<0.05, *** p<0.001, **** p<0.0001, n=3.

FIG. 8: Human al-antitrypsin detected in rat plasma after HMB002 transplantation. Animals were transplanted with alginate microbeads containing HC with or without MSC in SLG20 or MVG GRGDSP alginates. Results are shown as mean±SEM. ** p<0.01, **** p<0.0001, n=3.

FIG. 9: Weight gain in Sprague Dawley rats transplanted with HMB002.

In FIGS. 10 to 25, lines represent the mean values and dots the individual results. Grey dotted lines represent the upper and lower bounds of the reference ranges for Sprague Dawley rats. Some of the parameters analysed showed values slightly different from the reference ranges. However, this may be due to minor differences in the outbred animals used.

FIG. 10: Prothrombin time in Sprague Dawley rats transplanted with HMB002.

FIG. 11: White blood count in Sprague Dawley rats transplanted with HMB002.

FIG. 12: Red blood count in Sprague Dawley rats transplanted with HMB002.

FIG. 13: Haemoglobin levels in Sprague Dawley rats transplanted with HMB002.

FIG. 14: Haematocrit (PCV) in Sprague Dawley rats transplanted with HMB002.

FIG. 15: Mean red blood cell volume (MCV) in Sprague Dawley rats transplanted with HMB002.

FIG. 16: Mean corpuscular haemoglobin (MCH) in Sprague Dawley rats transplanted with HMB002.

FIG. 17: Mean corpuscular haemoglobin concentration in Sprague Dawley rats transplanted with HMB002.

FIG. 18: Platelet count in Sprague Dawley rats transplanted with HMB002.

FIG. 19: Mean platelet cell volume in Sprague Dawley rats transplanted with HMB002.

FIG. 20: Urea levels in Sprague Dawley rats transplanted with HMB002.

FIG. 21: Aspartate aminotransferase (AST) activity in Sprague Dawley rats transplanted with HMB002.

FIG. 22: Alanine aminotransferase (ALT) activity in Sprague Dawley rats transplanted with HMB002.

FIG. 23: Creatinine levels in Sprague Dawley rats transplanted with HMB002.

FIG. 24: Sodium levels in Sprague Dawley rats transplanted with HMB002.

FIG. 25: Potassium levels in Sprague Dawley rats transplanted with HMB002.

FIG. 26: Immunogenicity study in vitro. Levels of activation of lymphocytes shown as % CD69 positive cells. Results are shown as mean±SD, n=3. (TH, T helper; TC, T cytotoxic; NK, natural killer cells).

FIG. 27: HMB002 appearance at retrieval after transplantation in Sprague Dawley rats. Images of microbeads retrieved from peritoneal cavity of Sprague Dawley rats 28 days after transplantation. Hepatocytes and MSC co-encapsulated in MVG GRGDSP (HMB002) (A), SLG20 (B) or LMG GRGDSP (C) alginate microbeads. All images were taken using a Leitz Diavert inverted microscope on fields randomly selected. Magnifications were 4× and 10× for images shown on the left-hand or right-hand side, respectively.

FIG. 28: Alginate disk comparison—Hepatocytes were encapsulated in a newly developed high throughput system (disks in 96 well plates), or as a control, cultured in 2D on plastic/collagen (black and grey bars), with or without MSCs and kept in culture for up to 3 weeks. Whilst there was little difference in cell function for the first few days of culture in microdisks (data not shown), after 2 weeks there was a clear distinction between the cells encapsulated in the 2 alginates containing RGD motifs (green and red bars) as compared to the all the other ones, REDV, VAPG and the SLG alginate devoid of any anchoring peptide. ** p<0.01; ***p<0.001.

FIG. 29: data on importance of hepatocyte/MSC cell ratio: Hepatocytes were encapsulated in a newly developed high throughput system (disks in 96 well plates), with varying ratios of MSCs and kept in culture for up to 3 weeks. Albumin and alpha-1 antitrypsin where then analysed using an ELISA assay. Increasing the quantity of MSCs clearly improved the hepatocyte-specific function in the microdisks, with a 3:1 ratio deemed overall optimal.

FIG. 30: Comparison of the function of cells in HMB002 (G-RGD MBs), Sigma alginate microbeads or depolymerised-centre APA Sigma alginate microbeads. Hepatocytes and MSCs were encapsulated with a 2:1 ratio (i) following Shi's protocol (APA Sigma alginate) to generate microbeads with a depolymerised centre, (ii) using a simple encapsulation method in the same Sigma alginate, or (iii) in the G-RGD peptide alginate used to produce HMB002 microbeads. The cell functions were assessed using an MTT assay, a detection of liver-specific albumin or α-1 antitrypsin (AAT), as well as ureogenesis.

FIG. 31: Optimisation of HMB002 microbead cryopreservation. HMB002 microbeads were cultured in various preconditioning media containing platelet lysate (PL, 5%), human albumin solution (HAS, 10%) with or without trehalose (Treh, 0.1M), for 20 hr, before being cryopreserved in UW supplemented with 10% DMSO and 5% glucose. Cell viability was analysed using FDA fluorescence, shown here at day 6 after thawing (Panel A=Control (no pre-treatment), B=E1 (PL), C=E2 (PL+Treh), D=E3 (HAS) and E=E4 (HAS+Treh)) and further analysed with Image J. Synthetic functions were analysed by ELISA detecting A1AT production by the encapsulated hepatocytes. The combination of trehalose and platelet lysate in the preconditioning medium resulted on the best cell viability and function once the microbeads were thawed.

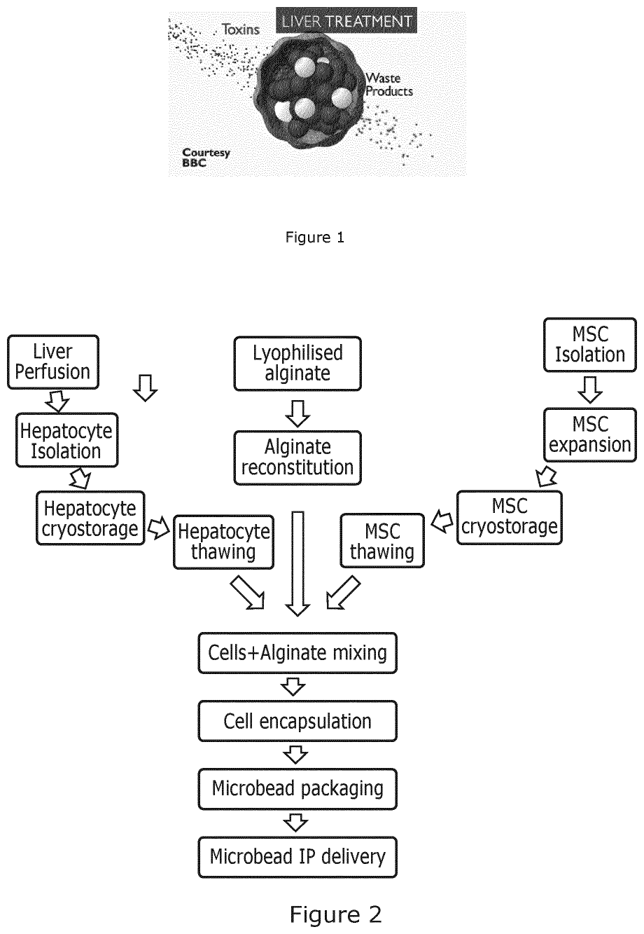

### EXAMPLES

The present invention will now be described with reference to the following non-limiting Examples.

### Example 1: Preparation of Encapsulated Cells—HMB002

An alginate solution was prepared by dissolving 100 mg sodium RGD-alginate (NOVATACH MVG-GRGDSP cat. #4270129) in 9.1 ml sterile saline to obtain a 1.1% alginate solution. The solution was left overnight to dissolve using a magnetic stirrer at room temperature. Best results were obtained when the alginate solution was kept overnight at 2-8° C. after resuspension. Freshly thawed human hepatocytes (HC) and early passage umbilical-cord derived mesenchymal stromal cells (MSC) were resuspended in a suitable cell culture medium (e.g., Minimum Essential Medium—Eagle with Earle's BSS). The cells were then pelleted and mixed in the alginate just before encapsulation, using 2.5×10⁶ hepatocytes and 0.8×10⁶ MSC per ml of alginate. The encapsulation was done using a Buchi encapsulator, with a 200-300 μm nozzle, and calcium chloride as gelation solution (100 mM CaCl₂ in sterile 0.9% NaCl). The gelation process lasted 10 min after which the cells were rinsed twice in 0.9% NaCl. The medium was then replaced with transplant medium for subsequent clinical use of the HMB002 ATMP.

### Example 2: Comparison of Hepatocyte Function in Various Types of Peptide Alginates

The binding of peptides to the alginate backbone is meant to mimic natural protein structures where cells usually anchor, which would promote better cell function upon encapsulation. As described in Table 2 below, RGD, VAPG and REDV are peptides found in liver proteins (Fibronectin, Vitronectin, Laminin, Collagen, Elastin) which therefore appear like ideal anchorage motifs for liver cells such as hepatocytes. To determine the optimal anchorage peptide, alginate solutions were prepared by dissolving 100 mg sodium RGD-alginate (NOVATACH MVG-GRGDSP cat. #4270129); 100 mg sodium SLG20-alginate (NOVATACH SLG20 cat. #4202021); 100 mg M REDV (NOVATACH M REDV cat. #4270421); or 100 mg G VAPG (NOVATACH G VAPG cat. #4270220) in 9.1 ml sterile saline to obtain a 1.1% alginate solution.

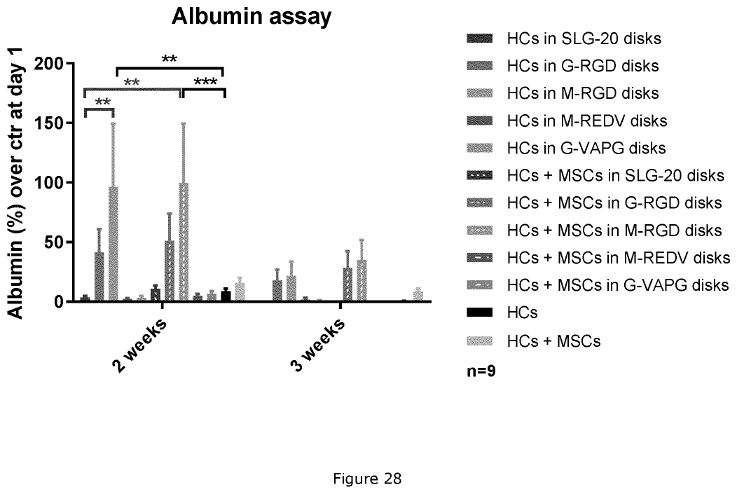

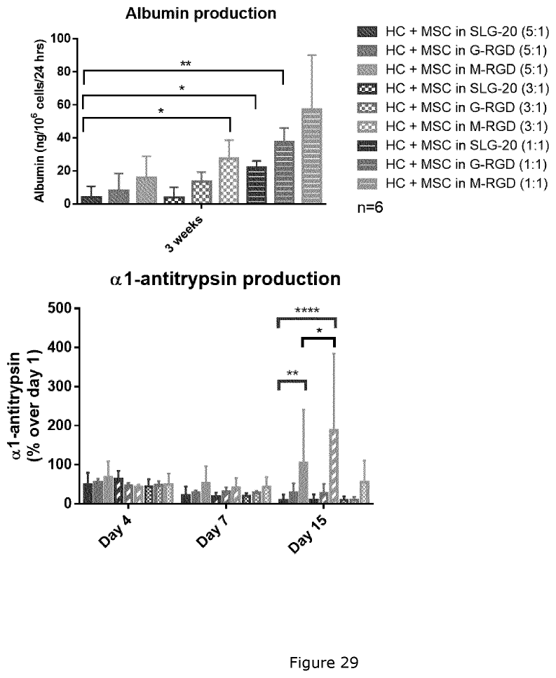

To simplify the comparison, we prepared a high throughput in situ cross-linked alginate microdisks (MDs), using an internal gelation strategy with some modifications. Briefly, alginate solution in 0.9% NaCl was mixed with cells (same density as in the microbeads) in the presence of an aqueous suspension of CaCO₃ (Sigma Aldrich) and D-glucono-delta-lactone (GDL, Sigma Aldrich). The pH was kept neutral by adjusting the molar ratio of CaCO₃/GDL. Gelation was obtained at 37° C. for 20 min. The same volume of microdisks (50 μL) was used in each well of a 96-well microplate. The albumin production results clearly show that alginates with an RGD peptide (both MVG GRGDSP and LVM GRGDSP) allow a much better cell function than the VAPG or REDV peptide alginates (FIG. 28).

TABLE 2
Peptide-Coupled Alginates Tested for Cell Encapsulation

| | | Peptide | ECM protein | G/M |
|---|---|---|---|---|
| Alginate | Abbreviation | sequence | mimic | Ratio |
| NOVATACH ™ | MVG | GRGDSP: | Fibronectin, | ≥1.5 |
| MVG GRGDSP | GRGDSP | Glycine (G), | Vitronectin, | |
| NOVATACH ™ | LVM | Arginine (R), | Laminin, | ≤1 |
| LVM GRGDSP | GRGDSP | Glycine (G), | Collagen | |
| | | Aspartate (D), | | |
| | | Serine (S), | | |
| | | Proline (P) | | |
| NOVATACH ™ | G VAPG | VAPG: Valine | Elastin | ≥1.5 |
| G VAPG | | (V), Alanine | | |
| | | (A), Proline | | |
| | | (P), Glycine | | |
| | | (G) | | |
| NOVATACH ™ | M REDV | REDV: | Fibronectin | ≤1 |
| M REDV | | Arginine (R), | | |
| | | Glutamic acid | | |
| | | (E), Aspartic | | |
| | | acid (D), | | |
| | | Valine (V) | | |
| PRONOVA ™ | SLG20 | No peptide | n/a | ≥1.5 |
| UP SLG20 | | modification | | |

The sequences of peptides covalently attached to the alginate are described with the proteins of the extracellular matrix (ECM) where these peptides are most commonly found. The G/M ratio is described and indicates the composition of each alginate in terms of α-L-guluronate (G) and β-D-mannuronate (M) residues. G residues are predominantly involved in the gelation process, therefore the alginates with a G/M ratio higher than 1.5 are usually stiffer than the ones with a G/M ratio lower than 1.

### Example 3: Comparison of HMB002 Microbeads and Alginate/Poly-L-Lysine/Alginate (APA) Depolymerised Centre Microbeads

By way of comparison, beads of hepatocytes and MSCs were prepared using the depolymerisation method described by Shi et al., 2009 (Transplantation: Nov. 27, 2009-Volume 88-Issue 10-p 1178-1185; doi: 10.1097/TP.0b013e3181bc288b). The depolymerisation of the microbead's core is meant to allow the cells to interact more efficiently, leading to a better microbead function. For the production of alginate/poly-L-lysine/alginate (APA) microbeads, hepatocytes and MSCs, in a ratio of 2:1 were mixed with an equal volume of sodium alginate (Sigma), and the resultant suspension extruded through a droplet generator and sprayed into 100 mM CaCl₂ solution to form beads. The beads were washed, treated with 1% poly-L-lysine to create a shell around the beads, rewashed before being incubated in 50 mM sodium citrate solution for 10 minutes to dissolve the alginate cores. Eventually, the capsules were then treated with alginate to create a new external layer of alginate.

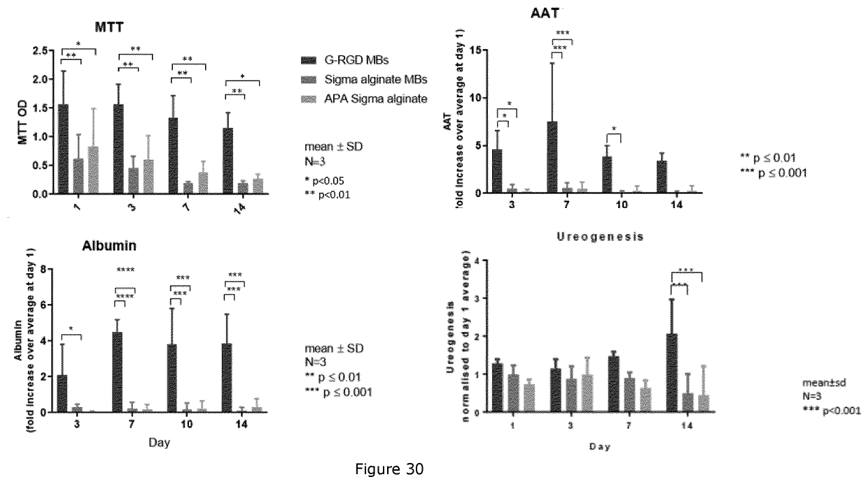

The hepatocyte functions were analysed in the APA beads and compared to the HMB002 microbeads—modified to have the same ratio of hepatocytes and MSCs as in Shi's study—as well as simpler, non-depolymerised microbeads made of the same Sigma alginate, all in triplicate, for up to 2 weeks. Hepatocytes and MSCs in HMB002 microbeads displayed a much higher oxidative function, assessed through an MTT assay, than in the two other microbead systems, on all days when they were tested (p<0.05 vs the depolymerised core microbeads, see FIG. 30). Albumin production, a liver-specific function, was 10 to 28-fold higher in the HMB002 microbeads than in the APA beads, depending on the day of analysis (see FIG. 30). A1AT, another protein produced exclusively by hepatocytes in our system was strongly produced from HMB002 microbeads, up to 60-fold higher than from depolymerised core APA microbeads. Ureogenesis was similar in all 3 bead types, except at day 14 when it was higher in HMB002 beads than any of the two other types of microbeads. In conclusion, the HMB002 is far superior to simple, or APA beads, made of hepatocytes and MSCs, in terms of function.

### Example 4: Primary Pharmacodynamics

### A. In Vitro Studies

Human primary HC and MSCs were co-encapsulated in five different peptide-alginates (shown in Table 2 below) using an in-situ crosslink approach, with production of microdisks that had shown to satisfactorily mimic microbead encapsulation (Iansante et al., 2018, Front Med (Lausanne) 5:216).

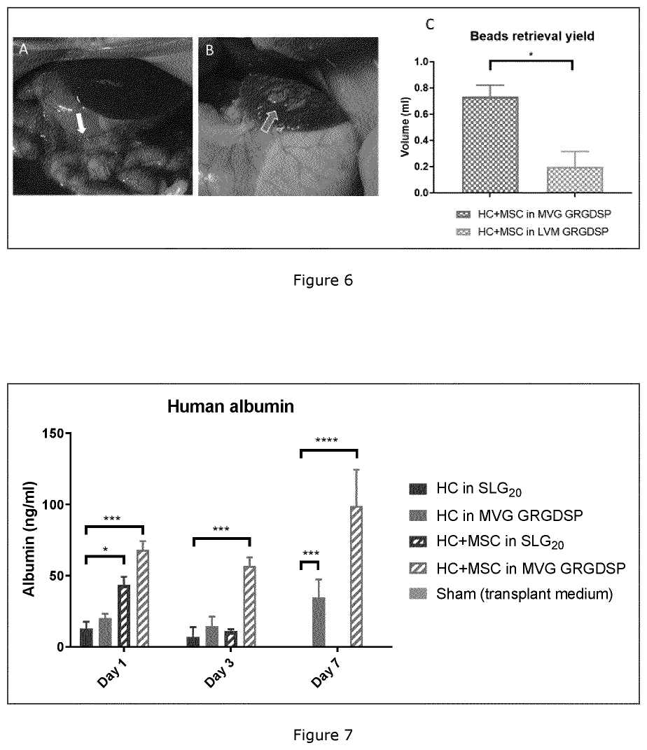

This preliminary pre-screening showed that encapsulation of HC and MSC at a ratio of 3:1 in GRGDSP-coupled alginates (MVG GRGDSP and LVM GRGDSP) improved the capacity of hepatocytes to produce albumin, one of the main markers commonly measured to analyse hepatocyte functions. Therefore, HC and MSC were co-encapsulated at a 3:1 ratio in each of the two GRGDSP-coupled or SLG20 alginate microbeads. A preliminary test in vivo in Sprague Dawley rats by intraperitoneal transplantation showed that LVM GRGDSP microbeads partially dissolved within 28 days (FIG. 6), while MVG GRGDSP microbeads did not show any change in size and shape. Therefore, MVG GRGDSP was used for all the following tests and eventually became the alginate of choice for the HMB002 product. HMB002 microbeads comprise primary human hepatocytes (HC) and mesenchymal stromal cells (MSCs) encapsulated in MVG GRGDSP alginate.

### B. Cell Viability—MSC & HC

### Methods

Cell viability/function was determined using a calcein-AM assay. Briefly, supernatant was removed, and microbeads were depolymerised in situ by addition of 50 mM ethylenediaminetetraacetic acid (EDTA, Sigma Aldrich). Cells were recovered by centrifugation and incubated with 4 M calcein-AM (Santa Cruz) in a black 96-well plate. As calcein-AM penetrates the cells and is converted into green fluorescent calcein by cellular esterases present only in viable cells, cell viability was assessed by measuring the green fluorescence emitted by the cells every minute for 30 minutes using FLUOstar Omega plate reader (BMG Labtech, Aylesbury, United Kingdom). Results were represented as relative fluorescence units (RFU)/minute.

A viability staining was also performed. Briefly, microbeads (50 μl) were washed with Dulbecco's phosphate-buffered saline (DPBS, Gibco), and stained with 10pg/mL fluorescein diacetate (FDA, Sigma-Aldrich) and 20pg/mL propidium iodide (PI, Sigma-Aldrich), and then visualised with a fluorescent microscope (Leica Microsystems Ltd, Milton Keynes, UK). Live cell cytoplasm appeared bright green (FDA), while dead cells, whose membrane had become permeable, had bright red-stained nuclei (PI).

### Results

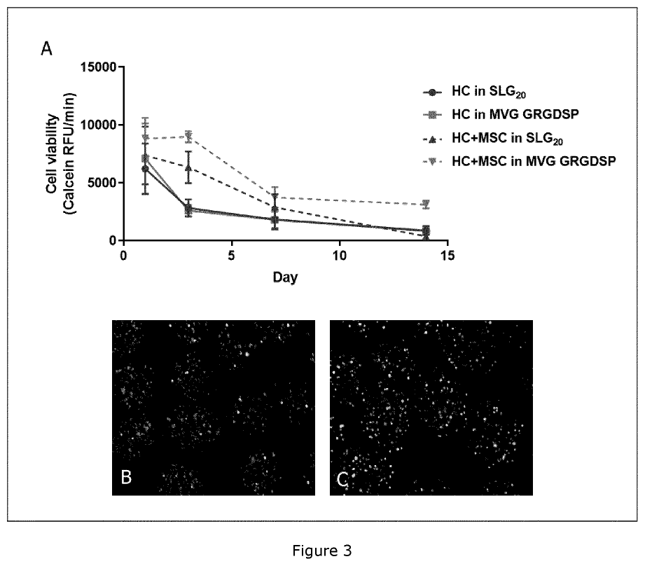

The analysis of microbeads by Calcein-AM assay showed similar cell viability in HC encapsulated in SLG20 and MVG GRGDSP alginate. HC co-encapsulated with MSC in MVG GRGDSP alginate show a higher viability than with SLG20 alginate (FIG. 3A). This result was confirmed by staining HC microbeads with FDA/PI 9 days after production, with an overall better cell viability shown for the MVG GRGDSP than in the SLG20 microbeads (FIG. 3, B and C).

### C. Hepatic-Specific Functions

The liver is an essential organ in the human body, responsible for many vital functions. Hepatocytes are the chief functional cells of the liver and perform an astonishing number of metabolic, endocrine and secretory functions.

The most common markers of hepatic functions measured in vitro and used in this study are i) biosynthetic capacity, assessed by measurement of proteins typically produced by hepatocytes, i.e. albumin and al-antitrypsin (AAT); ii) metabolic capacity, assessed by measurement of hepatocyte's capacity to detoxify ammonia through the urea cycle; iii) xenobiotics detoxifying capacity, assessed by phase 1 and 2 activity.

Phase 1 metabolism results in small chemical changes that make a compound more hydrophilic, so it can be effectively eliminated by the kidneys. These reactions usually involve hydrolysis, oxidation or reduction mechanisms and happen in the hepatocytes. The enzymes responsible for most phase 1 reactions belong to a large family of inducible enzymes, called cytochrome P450. The P450 superfamily is divided into families (e.g. CYP1, CYP2, CYP3, etc.) and in subfamilies (labelled with letters A, B, C, etc). In man, more than 50 isoforms have been isolated with members of the CYP450 families 1, 2 and 3 being the main factors responsible for the metabolism of drugs and other xenobiotics. Among these isoforms, CYP1A1/2 and CYP3A4 are often used as markers of hepatocyte functions in vitro, as they are responsible of a large number of phase 1 metabolisms in humans.

Phase 2 metabolism takes place if phase 1 is insufficient to clear a compound from circulation, or if it generates a reactive metabolite. These reactions usually involve adding a large polar group (conjugation reaction), such as glucuronide, to further increase the compound's solubility. Many enzymes are involved in the phase 2 metabolism, most of them in the liver. Considering the important role of phase 2 activity in vivo, it is usually detected as part of the in vitro tests to measure hepatocyte functions.

All key markers of hepatocyte functions were measured on the microbeads to see whether they would be able to provide an adequate metabolic function.

### Methods

Human albumin and al-antitrypsin secreted by microbeads in the culture medium over 24 hours were quantified using a specific Enzyme Linked Immunosorbent Assays (ELISA) Quantitation Kit, respectively (Bethyl Laboratories, Tx, USA).

For urea synthesis measurement, microbeads were washed twice with phosphate-buffered saline (PBS, Gibco) and incubated with 5 mM ammonium chloride in serum-free medium for 6 hours. The supernatant was collected and analysed using a QuantiChrom Urea Assay Kit (Universal Biologicals, Cambridge, UK), according to the manufacturer's instructions.

Hepatic drug metabolism was assessed by measuring the activity of CYP1A1/2 and 3A4. CYP1A1/2 inducibility was assessed using the ethoxyresorufin O-deethylation (EROD) assay. For this, microbeads were treated for 72 hours with omeprazole (50 μM), and their ability to metabolise 7-ethoxyresoryfin to fluorescent resorufin was measured using a plate reader. A resorufin standard curve was used to express the results as ng of resorufin formed per hour. CYP3A4 activity was measured using a commercial P450-Glo™ cell-based assay (Promega). Cell microbeads were treated with 25 μM rifampicin for 72 hours to induce CYP3A4, followed by incubation with a luminogenic CYP3A4 substrate. The signal was measured with a plate reader and expressed as relative luminescence units (RLU)/minute.

Phase 2 activity was determined by the measurement of resorufin metabolism. Microbeads were incubated with 50 ng/mL of resorufin for 2 hours at 37° C. Once resorufin is conjugated by phase 2 activity enzymes, it loses its fluorescence. Therefore, phase 2 metabolism is measured as a decrease in resorufin fluorescent signal and expressed as ng of conjugated-resorufin formed per hour.

### Results

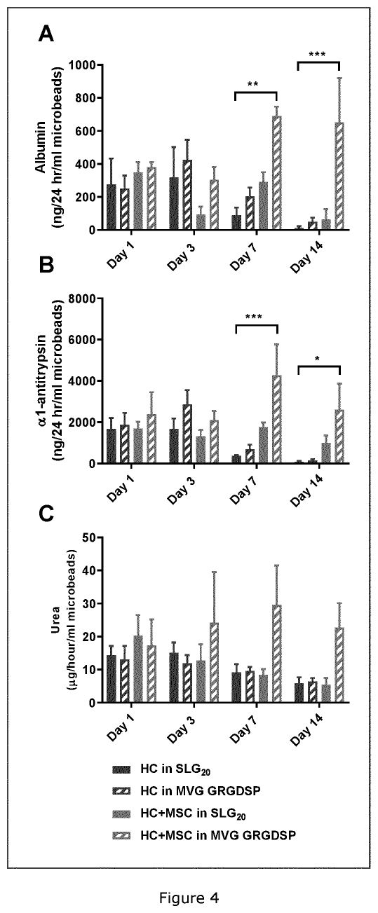

Albumin release in cell culture medium is significantly higher when HC are co-encapsulated with MSC in MVG GRGDSP alginate compared to SLG20 (see FIG. 4A, day 7 and 14). Similarly, the production of α1-antitrypsin (AAT), another common marker of hepatic functions, is significantly improved when hepatocytes are co-encapsulated with MSC in MVG GRGDSP alginate microbeads, as shown in FIG. 4B (day 7 and 14).

The production of urea was not significantly different in the groups analysed (FIG. 4C).

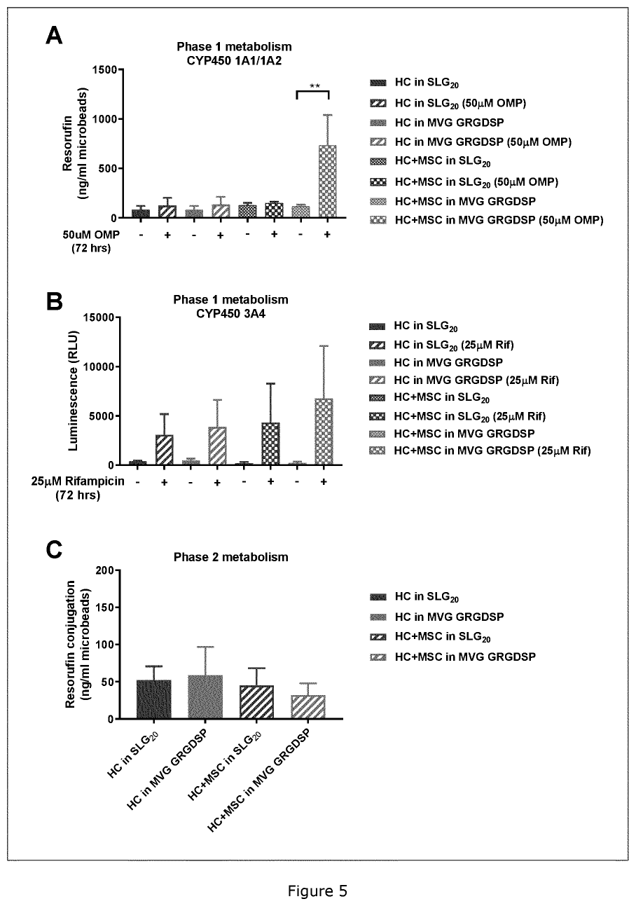

Hepatocytes have an important detoxifying role in the organism, mainly mediated by phase 1 and 2 enzymes. Therefore, it is highly important that microbeads show some activity of these enzymes to ensure an efficient metabolic function. CYP1A1/1A2 activity was measured at day 4 after 72-hour treatment of the microbeads with omeprazole, a known inducer of these isoforms. The treatment with omeprazole significantly increased CYP1A1/1A2 activity only when hepatocytes were co-encapsulated with MSC in MVG GRGDSP alginate microbeads (FIG. 5A). Also tested was the ability of the cells to induce CYP3A4, treating the microbeads with rifampicin for 72 hours. All the conditions tested showed a similar induction, although without any statistically significant difference

(FIG. 5B). Similarly, the analysis of phase 2 metabolism resulted in similar functions in the conditions analysed (FIG. 5C).

### D. In Vivo Studies

Microdisk high throughput experiments demonstrated that the two GRGDSP-coupled alginates (MVG GRGDSP and LVM GRGDSP) showed the best results as compared to all the modified alginates tested, with little differences. Consequently, as a preliminary experiment, Sprague Dawley rats were transplanted intraperitoneally with microbeads containing HC and MSC encapsulated in either MVG GRGDSP or LVM GRGDSP alginate. The animals were monitored, and microbeads retrieved after 28 days. It was observed that LVM GRGDSP alginate microbeads partially dissolved within 28 days (FIG. 6), while MVG GRGDSP microbeads did not show any change in size and shape. In addition, the analysis of the retrieved microbeads showed a significant lower volume of LVM GRGDSP microbeads (0.2+0.1 mL) compared to MVG GRGDSP (0.7+0.1 mL). Consequently, LVM GRGDSP was deemed inappropriate and MVG GRGDSP alginate was selected as the encapsulation excipient for HMB002.

Metabolic function of cells in HMB002 was investigated in vivo by transplanting Sprague Dawley rats intraperitoneally with SLG20 or MVG GRGDSP alginate microbeads containing human HC with or without human MSC (n=3 each group). Two negative controls were used, consisting in transplant medium (sham) and SLG20 empty microbeads (n=3 each group).

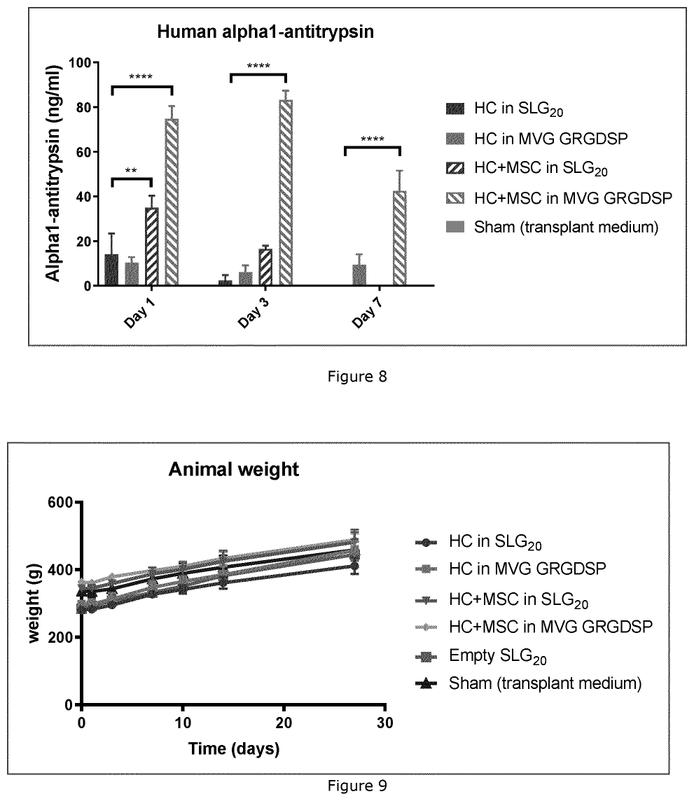

Plasma samples obtained from the animals were tested for human albumin and al-antitrypsin (AAT) levels using an ELISA approach, as described above (FIG. 7 and FIG. 8, respectively). Interestingly, human albumin was significantly higher in the animals injected with a combination of HC and MSC in MVG GRGDSP. Furthermore, at day 7 after transplantation, the level of albumin seemed to be increasing compared to earlier time points, suggesting that encapsulation in MVG GRGDSP alginate potentiates the effects of MSC on hepatocytes. These results are even more pronounced when considering the human al-antitrypsin production, where the addition of MSC to the MVG GRGDSP-encapsulated hepatocytes led to a 12-fold increase in AAT secretion at day 3, where it had only increased 4-fold in SLG20 microbeads. At day 7, the secretion of AAT was undetectable in cells encapsulated in SLG20, but was still steady in HC in MVG GRGDSP alginate and was even further increased with the inclusion of MSCs.

These results highlight that HMB002 microbeads have a significantly higher biosynthetic capacity than the other microbeads tested when transplanted in vivo.

### Example 4: Nonclinical Studies Overview

The study was done using human cells encapsulated in peptide alginate microbeads and infused in the peritoneal cavity of rats to easily detect human protein production by the hepatocytes and differentiate them from the animal's own liver function.

HMB002 microbeads comprise primary human hepatocytes (HC) and mesenchymal stromal cells (MSCs) encapsulated in MVG GRGDSP alginate. HMB001 comprise HC encapsulated in SLG20 alginate (no MSCs or peptide anchor).

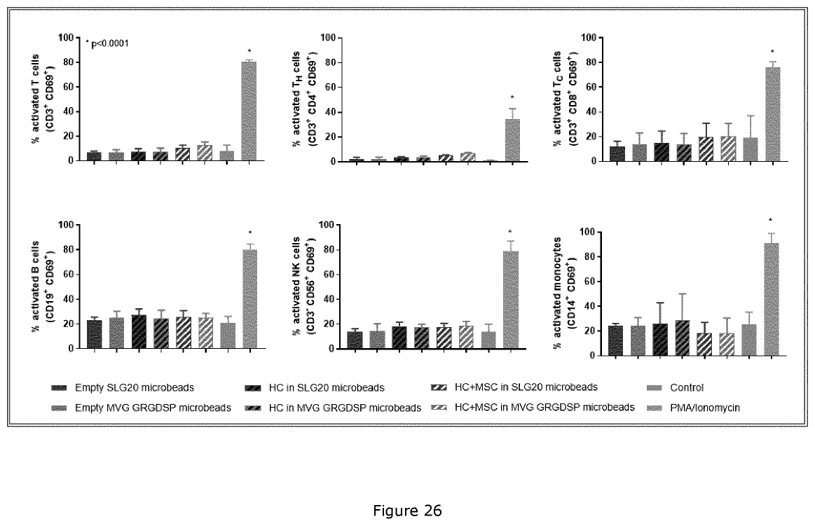

In preliminary in vitro studies, aimed at determining the optimal microbead matrix, HC and MSC were encapsulated in four different peptide-alginates (see Table 1 above) or in SLG20, which did not contain any peptide modification. This was done using a known in situ crosslink approach, with production of microdisks that had been shown to satisfactorily mimic microbead encapsulation. In vitro studies on the microdisks showed that when cells were encapsulated in the GRGDSP-modified alginates at a ratio of 3:1 (HC:MSC), hepatic functions were significantly improved compared to SLG20 alginate (Iansante et al., 2018, Front Med (Lausanne) 5:216; Iansante et al., 2016, Hepatology 64(1): 174A-5A). The results obtained on the microdisks were further confirmed on alginate microbeads, showing that the co-encapsulation of hepatocytes and MSC in MVG GRGDSP alginate microbeads (HMB002) significantly improved hepatic functions-albumin, α1-antitrypsin, urea production, phase 1 and 2 metabolic activity-compared to the hepatocytes encapsulated in SLG20 alginate (FIG. 4 and FIG. 5). In addition, immunogenicity studies were performed and showed that HMB002 did not trigger any immune cell activation when incubated with fresh human PBMCs in vitro (FIG. 26).

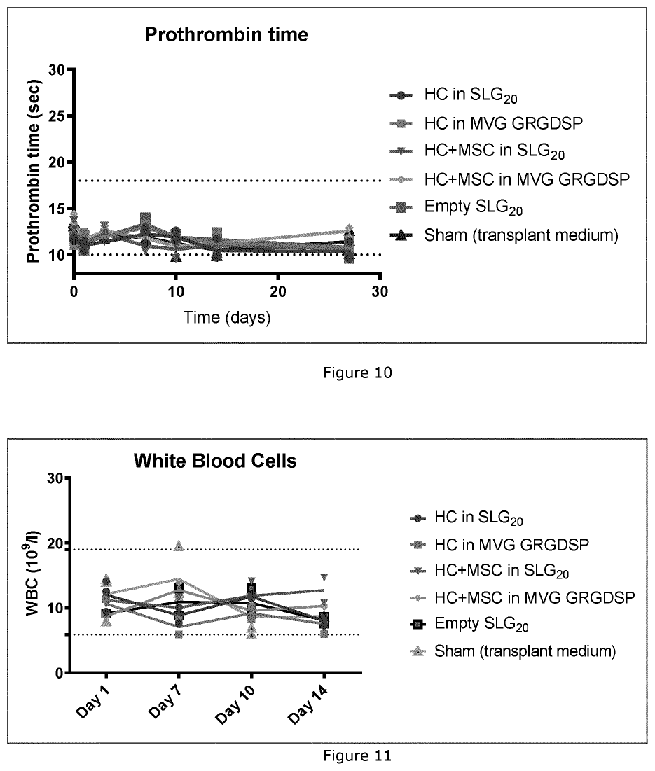

HMB002 was also tested in vivo by transplantation in Sprague Dawley rats. The microbeads were transplanted intraperitoneally and left in the animals for 4 weeks prior to retrieval. The rats were monitored and showed a healthy behaviour and normal weight gain (FIG. 9). In addition, full blood count, liver functions and main electrolytes were analysed and did not show any sign of toxicity (from FIG. 10 to 25).

The rats transplanted with HMB002 showed significantly higher levels of human hepatic proteins (albumin and al-antitrypsin, FIG. 7 and FIG. 8) in the bloodstream compared to HMB001 (i.e. microbeads containing only hepatocytes and in SLG20 alginate).

HMB002 may be particularly suited for the treatment of ALF in children, with the aim of bridging patients to full recovery or liver transplant. Preliminary data to support the bridging hypothesis have been obtained through results using HMB001, which contained donor-derived, adult hepatocytes alone, encapsulated in SLG20 alginate and used to treat eight children with ALF on a named patient basis, with promising results showing 50% surviving with their native liver and a further three patients bridged to transplantation.

Cell viability (for HC and MSC) and function (for HC) in microbeads have been assessed in vitro whilst the safety profile of HMB002 has been evaluated in a 28-day pharmacology study in normal rats. Data suggest that HMB002 in vivo is safe for the duration proposed in the clinical trial (28 days), with no treatment-related effects across a range of parameters. Microbeads were shown to be stable and secreted human protein in vivo in rats that were detectable for at least 7 days.

An immunogenicity study conducted with HMB002 in vitro suggests that the product will not be immunogenic.

### Example 5: Nonclinical Pharmacology

Previous results had shown that the co-culture of HC with MSC improves their survival and function in vitro. However, when co-encapsulated in SLG20 alginate, MSC were not as effective in terms of improving hepatocyte function.

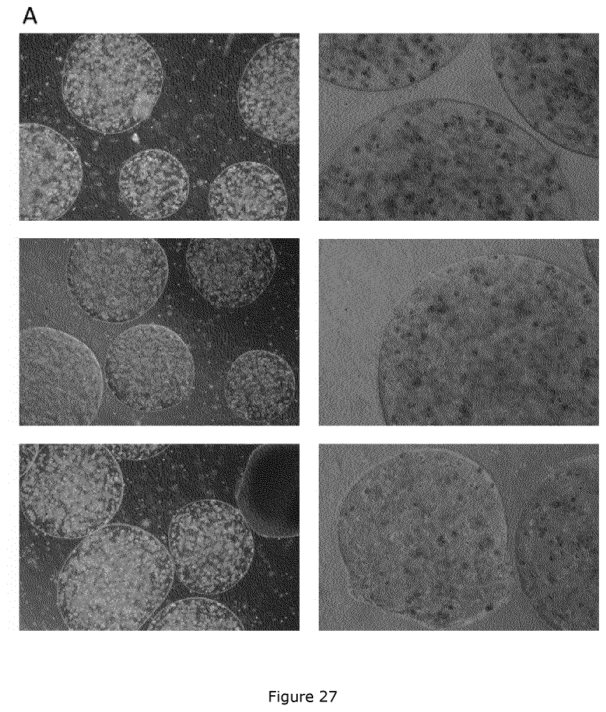

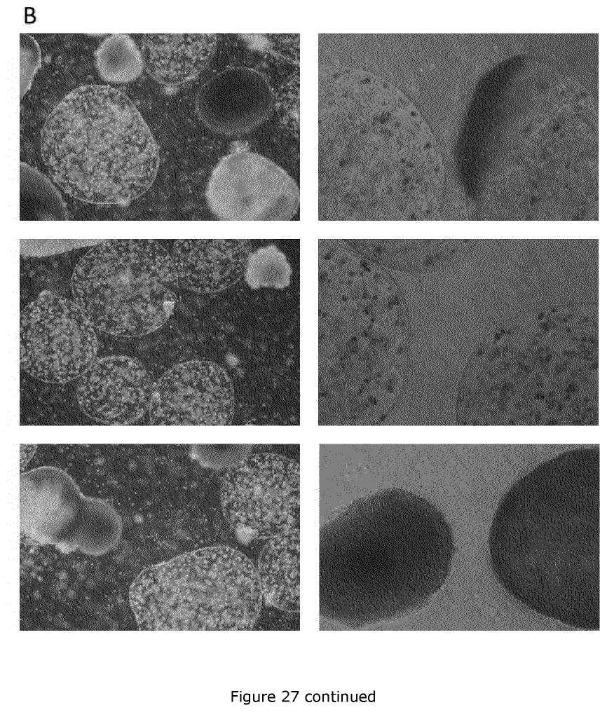

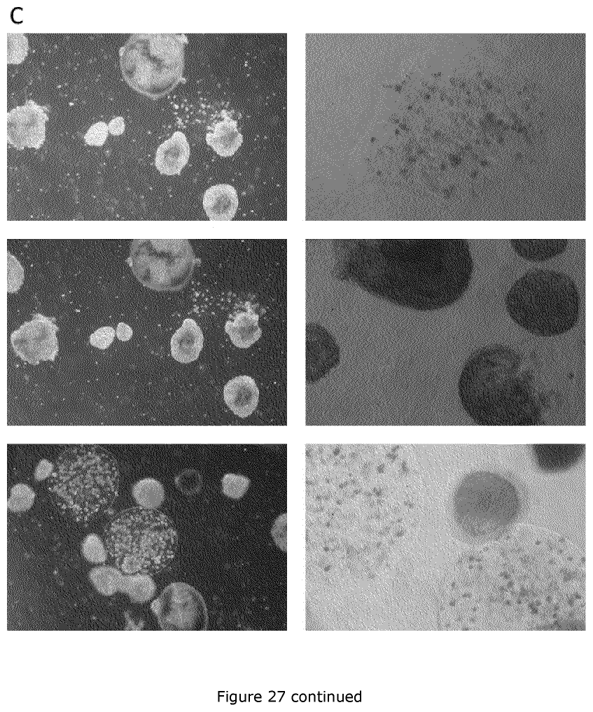

A range of peptide-coupled alginates (a mix of alginate coupled to a synthetic extracellular matrix (ECM) peptide) were compared to SLG20 alginate (see Table 1), to assess their ability to improve hepatocyte viability and function. In vitro, data indicate that RGD-coupled alginates (MVG GRGDSP and LVM GRGDSP, i.e. GRGDSP-coupled alginates with predominance of G and M residues, respectively) show significant improvement in HC function. Both MVG GRGDSP and LVM GRGDSP alginates were studied in vivo in rats, with MVG GRGDSP alginate proving to be superior to LVM GRGDSP (FIG. 6 and FIG. 27), thus becoming the alginate of choice for HMB002.

A range of in vitro and in vivo studies were performed to characterise the pharmacodynamics of HMB002, namely cell viability, biosynthetic capacity (albumin and al-antitrypsin production), urea synthesis, phase 1 and 2 hepatic drug metabolism in vitro, and biosynthesis of human hepatic proteins (albumin and al-antitrypsin) in vivo.

Data suggest that both HC and MSC in HMB002 remain viable following encapsulation. HC are active in key markers of hepatocyte function (biosynthetic, detoxifying and metabolic functions).

TABLE 3
List of Studies Investigating the Pharmacology of Alginate Encapsulated cells

| | In vitro/In vivo/ | Dose/Treatment/Route/ | Endpoint | |
|---|---|---|---|---|
| Study Number | Cells/Species/Strain | Duration | Measurement | Noteworthy Findings |
| VI39 | In vitro | 2.5 × 10⁶/ml HC in SLG20 vs | Cell viability, i.e. | HC encapsulated in MVG GRGDSP |
| | Cells: Human primary | MVG GRGDSP alginate | FDA/PI staining | alginate microbeads showed |
| | hepatocytes (HC186). | microbeads. | (day 9). | overall higher viability compared to |
| | | | | SLG20, 9 days after encapsulation |
| | | | | (FIG. 3). |
| VI53/VI57/VI59 | In vitro | 2.5 × 10⁶/ml HC ± | Cell | Cell viability was similar when HC |
| | Cells: Human primary | 0.83 × 10⁶/ml MSC | function/viability, | were encapsulated in SLG20 or |
| | hepatocytes (HC343, | encapsulated in SLG20 vs | i.e. Calcein-AM | MVG GRGDSP alginate microbeads, |
| | HC349, HC353, | MVG GRGDSP alginate | metabolism (day | however there was a tendency |
| | HC364); human | microbeads. | 1, 3, 7, 14); | towards higher viability when HC |
| | umbilical cord derived | Microbeads cultured up to | Albumin | were co-encapsulated with MSC in |
| | MSC (SW0G, VI2, | 14 days. | production (day | MVG GRGDSP alginate microbeads |
| | SW4). | | 1, 3, 7, 14); | (i.e. HMB002) (FIG. 3). |
| | | | α1-antitrypsin | The release of human albumin and |
| | | | production (day | α1-antitrypsin in cell culture |
| | | | 1, 3, 7, 14); | medium at day 7 and 14 was |
| | | | Urea production | significantly higher when HC were |
| | | | (day 1, 3, 7, 14) | co-encapsulated with MSC in MVG |
| | | | Phase 1 | GRGDSP alginate (i.e. HMB002) |
| | | | metabolic | compared to HC in SLG20 (i.e. |
| | | | activity: | HMB001) (FIG. 4). |
| | | | CYP1A1/1A2 | The activity of Cyp1A1/1A2 was |
| | | | activity (day 4, | significantly induced only in |
| | | | after induction | HMB002 (FIG. 5). |
| | | | with 50 μM | No differences were noticed in |
| | | | omeprazole for | terms of urea production, CYP3A4 |
| | | | 72 hours); | induction and phase 2 activity in |
| | | | CYP3A4 activity | the groups analysed (FIG. 5). |
| | | | (day 4, after | |
| | | | induction with | |
| | | | 25 μM rifampicin | |
| | | | for 72 hours); | |
| | | | Phase 2 | |
| | | | metabolic | |
| | | | activity, i.e. | |
| | | | resorufin | |
| | | | conjugation (day | |
| | | | 4). | |
| VI39 | In vivo | Alginate microbeads: | Human albumin | Human albumin (FIG. 7) and α1- |
| | Cells: human primary | 2.5 × 10⁶/mL HC ± | released in | antitrypsin (FIG. 8) levels |
| | hepatocytes (HC186); | 0.83 × 10⁶/mL MSC | bloodstream | detected at day 1, 3 and 7 in the |
| | human umbilical cord | encapsulated in SLG20 vs | (day 1, 3, 7, 14, | plasma of rats transplanted with |
| | derived MSC (VI2). | MVG GRGDSP alginate | 28); | HMB002 were significantly higher |
| | Animals: Sprague | microbeads. SLG20 empty | Human α1- | than the levels measured in the |
| | Dawley rats | microbeads as a control. | antitrypsin | animals transplanted with |
| | | In vivo transplantation: cell | released in | HMB001. No human proteins were |
| | | alginate microbeads were | bloodstream | detected afterwards. |
| | | resuspended in transplant | (day 1, 3, 7, 14, | At day 7 after transplantation, the |
| | | medium (2:1 v/v) and | 28). | level of human albumin detected |
| | | intraperitoneally | | seems to be increasing compared |
| | | transplanted in rats as a | | to earlier time points, though not |
| | | single dose (10 mL | | reaching any statistical significance |
| | | microbeads/kg). Negative | | (99 ± 25 vs 68 ± 6 ng/mL at day 7 |
| | | controls: transplant medium | | and 1, respectively). |
| | | (sham) and SLG20 empty | | |
| | | microbeads. | | |

### Example 6: Pharmacokinetics and Product Metabolism in Animals

HMB002 comprises HC co-encapsulated with MSC (3:1 ratio HC:MSC) in alginate and presented as microbeads suspended in transplant medium (1:1 ratio microbeads:medium). It is intended to be injected as 20 ml/kg body weight into the peritoneal cavity of an infant or child with ALF where it remains until removed. As such, it has not been possible to conduct classical pharmacokinetic studies, in agreement with Directive 2001/83/EC Part IV Annex I and Directive 2009/120/EC, section 4.3.2. Rather, supportive data have been derived from an in vivo study where HMB002 microbeads were injected intraperitoneally in rats to assess their longevity. Data from alginate microbeads retrieved after the animal culling, 28 days after transplantation, indicate that microbeads remain intact in the peritoneum for at least this time. All the microbeads retrieved looked similar to their original aspect in terms of size and cell number, in stark contrast to what had happened with another alginate for which most microbeads had degraded (FIG. 6). In addition, HMB002 microbeads appeared almost completely clear from any coating after retrieval, as opposed to microbeads made of SLG20 or another alginate that were partially or fully coated, presumably by the host's immune cells (FIG. 27).

### Analytical Methods and Validation

Sprague Dawley rats were euthanised 28 days after transplantation and inspected after a midline laparotomy. Photographs of the peritoneal cavity were taken, and microbeads recovered. The microbead volume was estimated using graduated conical tubes. Images of microbeads after retrieval were taken using a DSLR camera connected to an inverted microscope.

### Absorption and Pharmacokinetics

An in vivo experiment conducted in rats indicate that HMB002 microbeads remain intact in the peritoneum for at least 28 days (this corresponds to the end of the test period) and, therefore, should not be absorbed.

### Distribution

An in vivo experiment conducted in rats indicate that HMB002 is not distributed and microbeads seem to remain intact in the peritoneum for at least 28 days (this corresponds to the end of the study).

### Pharmacokinetic Drug Interactions

As HMB002 consists of human cells normally found within the human body, we expect them to metabolize any drug as the recipient's own liver would do.

### Conclusion

Although classical pharmacokinetics studies were not conducted, a single dose in vivo study in rats was performed with intraperitoneal transplantation of HMB002. HMB002 microbeads retrieved 28 days after transplantation did not show any signs of shrinkage or damage.

TABLE 4
List of Pharmacokinetic and Metabolism Studies Performed During the Development of HMB002

| | In vitro/In | | | |
|---|---|---|---|---|
| | vivo/Cells/ | | | |
| Study | Species/ | Dose/Treatment/ | Endpoint | |
| Number | Strain | Route/Duration | Measurement | Noteworthy Findings |
| VI39 | In vivo | Alginate microbeads: | Observation of | HMB002 remained intact in the |
| | Cells: human | 2.5 × 10⁶/mL HC ± | peritoneal cavity for | peritoneum for 28 days without |
| | primary | 0.83 × 10⁶/mL MSC | signs of alginate | any signs of alginate degradation |
| | hepatocytes | encapsulated in SLG20 vs | degradation or | (FIGS. 6 and 27) |
| | (HC186); | MVG GRGDSP alginate | fibrosis. | |
| | human | microbeads. | Volume/shape/size | |
| | umbilical cord | In vivo transplantation: | of microbeads | |
| | derived MSC | cell alginate microbeads | recovered. | |
| | (VI2). | were resuspended in | | |
| | Animals: | transplant medium and | | |
| | Sprague | transplanted | | |
| | Dawley rats | intraperitoneally in rats as | | |
| | | a single dose (10 mL | | |
| | | microbeads/kg). | | |

### Example 7: Toxicology

HMB002 will be injected as a single dose of 20 mL per kg body weight into the peritoneal cavity—i.e., 25 million hepatocytes and 8.3 million MSC/kg—in infants or children with ALF and is proposed to provide metabolic function. HMB002 will remain in the peritoneum until such time as the native organ regenerates or a suitable liver is accepted for transplantation.

Rather than using an animal model of ALF with syngenic cells, where the specific function of the encapsulated cells would be complex to determine, HMB002 were injected intraperitoneally in rats, with doses closely resembling the intended clinical usage.

Rats injected intraperitoneally with the microbeads survived for 28 days and gained weight comparably to control animals. Similarly, there were no significant differences between HMB002 rats and control rats across a range of parameters (i.e., prothrombin time, white blood cells, red blood cells, haemoglobin, haematocrit, mean red blood cell volume, mean corpuscular haemoglobin, mean corpuscular haemoglobin concentration, platelet count, mean platelet cell volume, urea, aspartate aminotransferase, alanine aminotransferase, creatinine, sodium, potassium).

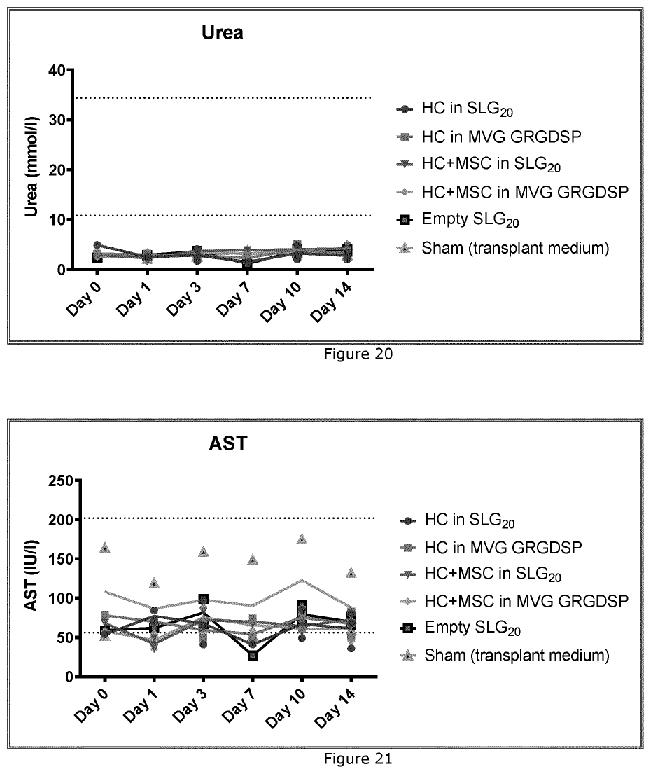

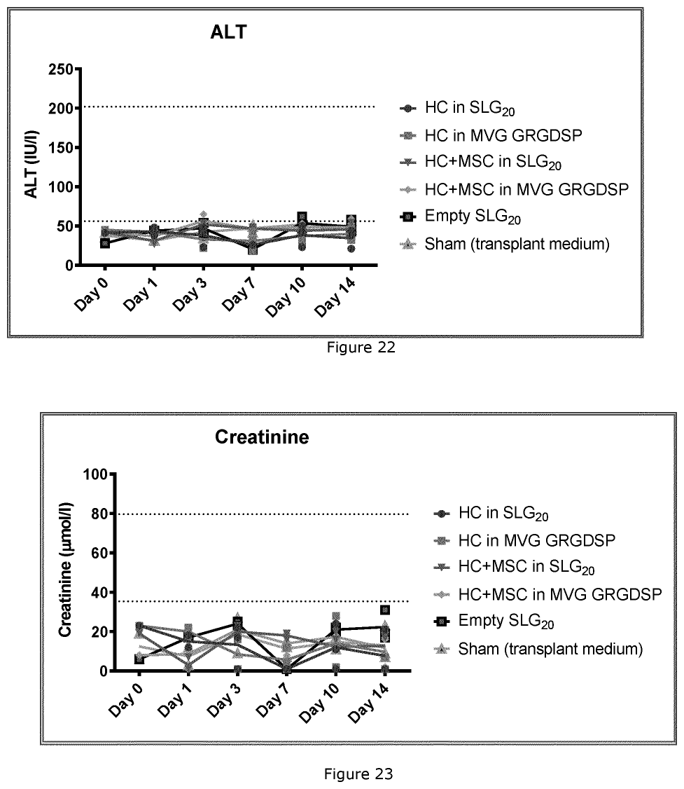

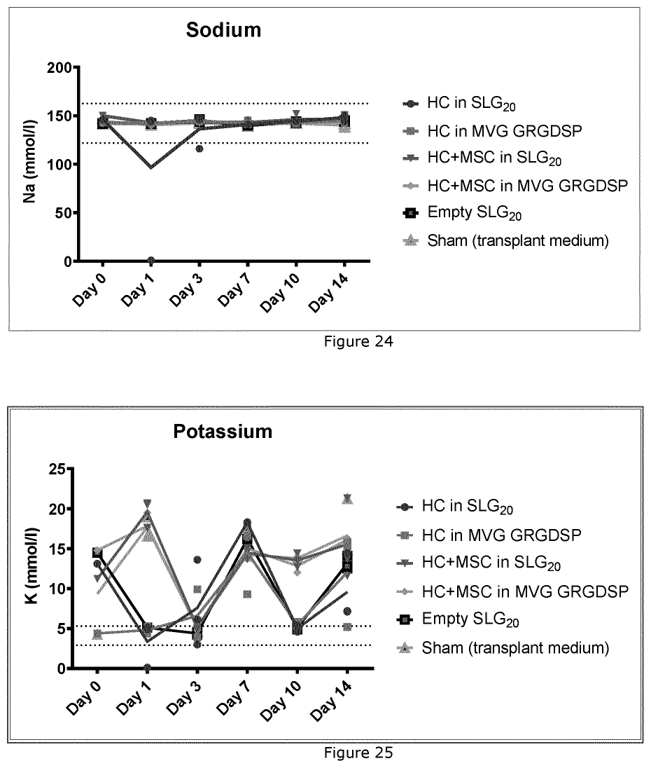

TABLE 5
List of toxicology studies performed during the development of HMB002

| | In vitro /In | | | |
|---|---|---|---|---|
| Study | vivo/Cells/ | Dose/Treatment/ | Endpoint | |
| Number | Species/Strain | Route/Duration | Measurement | Noteworthy Findings |
| VI39 | In vivo | Alginate microbeads: 2.5 × 10⁶/mL | Blood sampling and | All rats transplanted intraperitoneally |
| | Cells: human | HC ± 0.83 × 10⁶/mL MSC | weight measurement | with either HMB002 or the other |
| | primary | encapsulated in SLG20 vs MVG | were performed at | microbeads tested survived for 28 |
| | hepatocytes | GRGDSP alginate microbeads. | day 1, (3) 7, 10, and | days and gained weight comparably |
| | (HC186); | SLG20 empty microbeads as a | 14 after microbeads | to control animals (FIG. 9). |
| | human umbilical | control. | injection. | No variations in prothrombin time |
| | cord derived | In vivo transplantation: cell | Full blood count | were observed (FIG. 10). |
| | MSC (VI2). | alginate microbeads were | (white blood cells, | No variations in full blood count were |
| | Animals: | resuspended in transplant | red blood cells, | noticed (FIGS. 11-19) |
| | Sprague Dawley | medium (2:1 v/v) and | haemoglobin, | No significant differences in hepatic |
| | rats | intraperitoneally transplanted in | haematocrit, mean | and/or renal functions were observed, |
| | | rats as a single dose (10 mL | red blood cell | assessed by measurement of urea |
| | | microbeads/kg). Negative | volume, mean | (FIG. 20), aspartate |
| | | controls: transplant medium | corpuscular | aminotransferase (AST) (FIG. 21) |
| | | (sham) and SLG20 empty | haemoglobin, mean | and alanine aminotransferase (ALT) |
| | | microbeads. | corpuscular); plasma | (FIG. 22). |
| | | | analysis (sodium, | No significant variations in creatinine |
| | | | potassium, urea, | (FIG. 23) and sodium (FIG. 24) |
| | | | creatinine, aspartate | were registered. |
| | | | aminotransferase | Potassium levels showed high |
| | | | activity, alanine | variability and changes over time in |
| | | | aminotransferase | all the animals tested (including |
| | | | activity); | controls), probably due to samples' |
| | | | prothrombin time. | haemolysis (FIG. 25). |
| I47 | In vitro. | 2.5 × 10⁶/mL HC ± 0.83 × 10⁶/mL | Percentages of | None of the microbeads tested |
| | Cells: Human | MSC encapsulated in SLG20 vs | activated (CD69⁺) | showed any sign of immune cell |
| | primary | MVG GRGDSP alginate | lymphocytes, i.e. T | activation and immunogenicity |
| | hepatocytes | microbeads. Empty microbeads as | cells (CD3⁺CD69⁺), T | (FIG. 26). |
| | (HC186); | controls. | helper cells | |
| | human umbilical | Microbeads (500 μl) were | (CD3⁺CD4⁺CD69⁺), T | |
| | cord derived | incubated for 24 hours with PBMC | cytotoxic cells | |
| | MSC (SWOG): | (5 × 10⁵). PBMC were treated with | (CD3⁺CD8⁺CD69⁺), B | |
| | primary human | 500 ng/mL phorbol 12-myristate | cells (CD19⁺CD69⁺), | |
| | peripheral blood | 13-acetate (PMA) and 10 ng/mL | NK cells (CD3⁻ | |
| | mononuclear | ionomycin as a positive control of | CD56⁺CD69⁺), | |
| | cell (PBMC) from | lymphocytes activation. | monocytes | |
| | n = 3 donors. | PBMC were stained with | (CD14⁺CD69⁺). | |
| | | fluorescent-labelled antibodies | | |
| | | targeting CD3, CD4, CD8, CD14, | | |
| | | CD19, CD56, CD69 and analysed | | |
| | | by flow cytometry. | | |

### A. Single Dose Studies

Sprague Dawley male rats 8-10 weeks old and weighing between 200 to 300 g were injected intraperitoneally with SLG20 or MVG GRGDSP alginate microbeads containing human HC with or without human MSC (n=3 each group). Two negative controls were used, consisting in transplant medium (sham) and SLG20 empty microbeads (n=3 each group).

### Animal Follow-Up

### Methods

Animal sampling was done up to 28 days post transplantation. Blood sampling and weight measurement were performed at day 1, 7, 10, and 14 after microbead injection. Blood was collected in EDTA vials for full blood count analysis and lithium-heparin vials for plasma preparation. A few microliters of blood was used immediately for prothrombin time evaluation (CoaguChek® XS system, Roche Diagnostics). Full blood count and plasma analysis were performed by Viapath facility (King's College Hospital, London). Plasma analysis included quantification of sodium and potassium, urea, creatinine, conjugated bilirubin as well as aspartate aminotransferase (AST) and alanine aminotransferase (ALT) activities. Animals were euthanised at day 28.

### Results

All the rats injected intraperitoneally with the microbeads survived for 28 days and gained weight in a similar fashion (FIG. 9). Their aspect and overall behaviour support a healthy growth in all of them.

Increased prothrombin time is a sign of liver injury and it is commonly included in the diagnostic tests for acute liver failure. Therefore, it has been tested in the animals and showed no differences in the conditions tested (FIG. 10), all with values within the normal ranges (Petterino et al., 2006, Exp Toxicol Pathol. 57(3), pp. 213-9), as shown in the graphs (dotted lines).

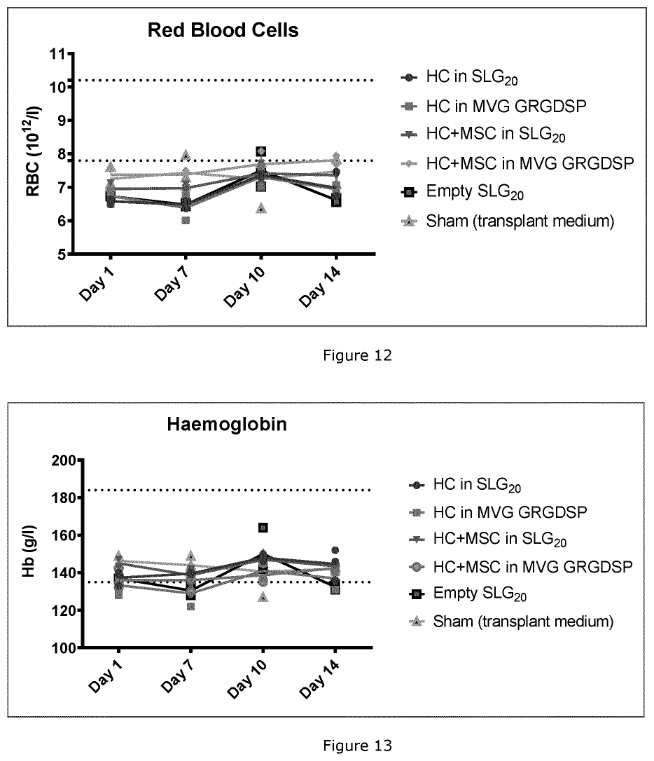

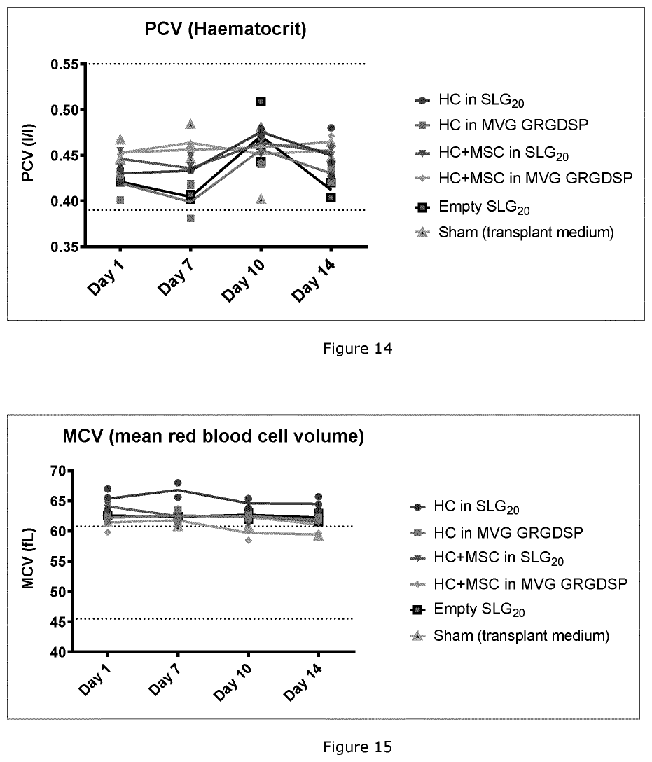

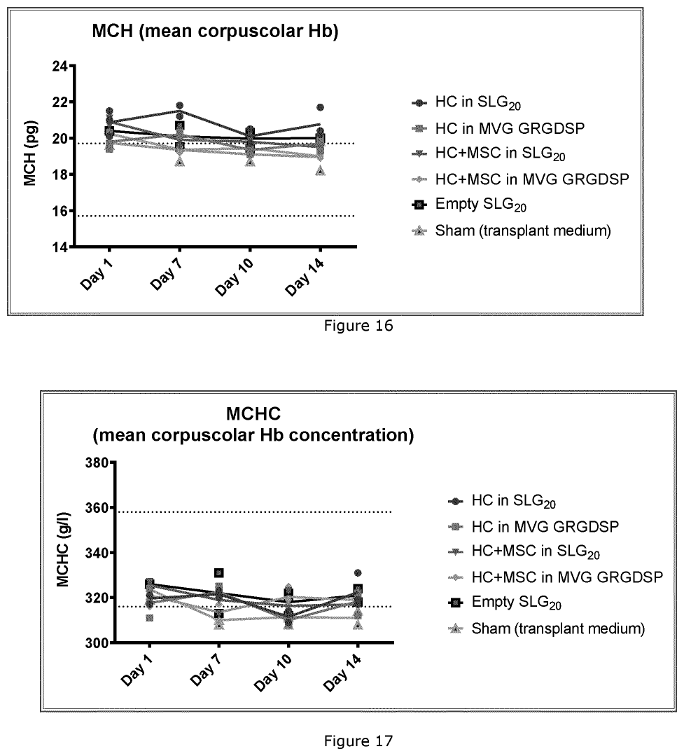

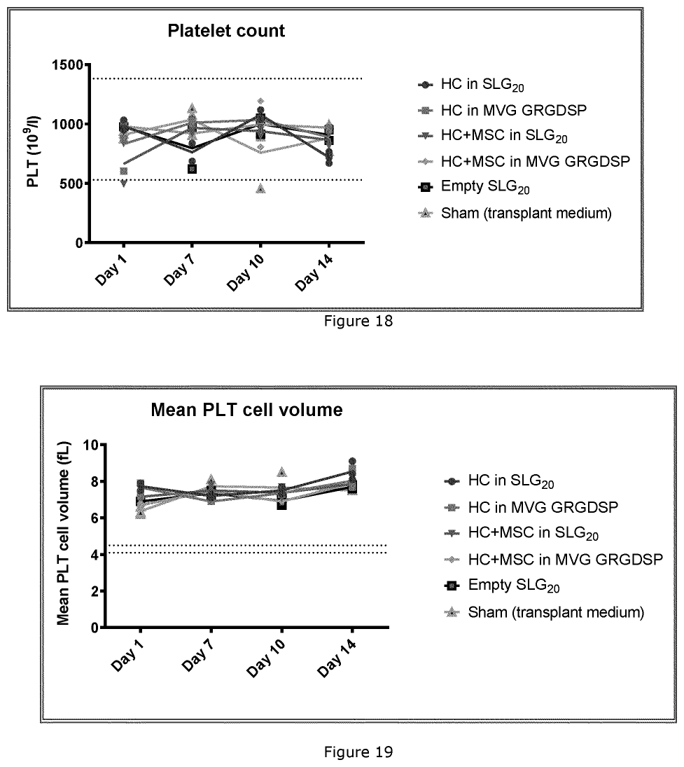

Full blood count was also performed at different time points on blood samples, analysing white blood cell (FIG. 11), red blood cells (FIG. 12), haemoglobin (FIG. 13), haematocrit (FIG. 14), mean red blood cell volume (FIG. 15), mean corpuscular haemoglobin (FIG. 16), mean corpuscular haemoglobin concentration (MCH) (FIG. 17), platelet count (FIG. 18), and mean platelet cell volume (FIG. 19). None of these parameters showed significant differences compared to the control groups. Some of the parameters analysed showed values slightly different from the reference ranges (shown in the graphs as grey dotted lines). However, this may be due to minor differences in the outbred animals used.

Other parameters were measured in the plasma of the rats to assess hepatic functions, including urea (FIG. 20), aspartate aminotransferase (AST) (FIG. 21) and alanine aminotransferase (ALT) (FIG. 22). Urea is produced primarily in the liver and excreted in the renal tubules. Therefore, the altered urea levels may reflect impaired function of the liver and/or kidneys. However, levels of urea in the animals tested did not show any significant change over time compared to the controls. As already seen for other blood parameters, the normal ranges are slightly higher than the values found in the literature, but this may depend on the animals used. AST and ALT massively increase in case of acute liver failure, due to necrosis of hepatocytes and release of their cytoplasm content. The detections of these two enzymes in blood samples are typical clinical measurement to detect any liver damage. ALT and AST did not show any significant difference in the different groups. However, one animal in the control sham group showed elevated values of AST. However, a raised level of AST was seen in this animal from day 0, so it does not seem to be related to the treatment Renal function has been assessed by measuring creatinine levels, without observing any differences between the different groups.

Finally, creatinine (FIG. 23) as well as sodium and potassium electrolytes were measured (FIG. 24 and FIG. 25 respectively). While creatinine and sodium did not show any significant difference in the groups tested, potassium levels showed high variability and changes over time. However, the measurement is highly dependent on the clarity of the plasma samples provided to the biochemistry lab and some of our samples were haemolysed which is then probably the cause of these erratic results.

### B. Local Tolerance

When HMB002 microbeads were transplanted in vivo in the peritoneal cavity of Sprague Dawley rats, they were well tolerated. At retrieval, they appeared to be confined in the peritoneum near the liver, intestine and mesentery (FIG. 27). There was no obvious signs of inflammation or fibrosis.

### C. In Vitro Immunogenicity

Biologics can elicit an immune response as they are potentially immunogenic. In general, the T-cell response drives the immunogenic reaction, which can be transient without clinical significance or lead to adverse effects. HMB002 microbeads are expected not to be immunogenic as the alginate behaves as a shield to the encapsulated cells. However, this was tested to see whether the microbeads are immunogenic and elicit the activation of immune cells in vitro when co-cultured with Peripheral Blood Mononuclear Cell (PBMC).

### Methods

Human hepatocytes alone or combined with MSC were encapsulated in SLG20 or MVG GRGDSP alginate. Empty microbeads produced using the same alginates were used for comparison. Microbeads (500 μl) were incubated for 24 hours with PBMC (5×10⁵) isolated from n=3 healthy donors. Pictures were then taken to investigate any sign of cell attachment to the microbeads and PBMC were stained with antibodies listed in Table 6 PBMC were treated with 500 ng/mL phorbol 12-myristate 13-acetate (PMA) and 10 ng/mL ionomycin as a positive control of cell activation.

### Results

None of the microbeads incubated with PBMC showed any sign of cell attachment when observed under the microscope after 24-hour incubation (data not shown). CD69 expression was used as a marker of lymphocyte activation. None of the conditions tested determined any significant cell activation compared to untreated PBMC, used as a control. By contrast, PMA and ionomycin treatment induced a significant activation of the cells in all the cell subsets analysed. These results indicate a lack of immunogenicity of both SLG20 and MVG GRGDSP alginates in vitro (FIG. 6).

TABLE 6
Antibodies used in immunogenicity study
Antibody

PE-conjugated anti-CD3
PE-CY-7-conjugated anti-CD4
FITC-conjugated anti-CD8
PE-CY-5-conjugated anti-CD56
APC-conjugated anti-CD69
APC-Cy7-Dead cell stain
FITC-conjugated anti-CD19
PerCP-conjugated anti-CD14

### Example 8: HMB002 Cryopreservation

HMB002 can be used directly after its production or be cryopreserved for long-term storage. The thawing of cryopreserved products is relatively straightforward. The cryopreservation will therefore also enable the shipment of the microbeads to remote places, using a dry shipper, where clinicians will be able to use them with very little training. The optimisation of the microbead cryopreservation is therefore an essential part of the development of HMB002 as a widely available treatment of liver diseases.

Cryopreservation protocols aim at preventing the formation of large ice crystals inside cells that would result in membrane damage and subsequent loss of cell viability and function. Two main strategies are used: 1/ the dehydration of cells using non-permeant hypertonic solutions and 2/ the use of intracellular compounds reducing the formation of ice crystals and stabilising the membrane structure. The speed at which the cells are frozen is also critical to prevent the formation of crystals, and led to the development of complex protocols using precise controlled-rate freezers. For cell therapy development, any product used must also comply with GMP regulations.

The hepatocyte and original microbead cryopreservation protocol commonly practiced uses the University of Wisconsin solution (UW) supplemented with typical components for cell freezing: glucose (5% W/V) and dimethyl sulfoxide (DMSO; 10% V/V) (Jitrarauch, S., et. al., Cell Transplantation. 2017:26 (8): 1341-1354). Therefore this solution has been used as the control in the microbead freezing optimisation process described below. Trehalose is a natural sugar made of two molecules of glucose and found in high concentration in diverse living organism-fungi, invertebrates, yeasts, insects, frogs-allowing them to sustain freezing temperatures. It has been successfully investigated as a cryoprotectant for numerous types of cells (red blood cells, hematopoietic stem cells, oocytes, sperm, Langerhans islets, mesenchymal stem cells, etc.). Trehalose has also showed some limited improvement of cryopreservation for primary hepatocytes (Miyamoto, Y. et. al., Cell Transplant. 2006; 15:911-919, Solocinski, J. et. al., Cryobiology 2017; 75:134-143, Katenze, E. et. al., Liver Transpl. 2007; 13: 38-45) However, the addition of trehalose to usual cryopreservation medium did not improve cell survival or function when the cells were encapsulated as they are in HMB002 (data not shown).

Platelet lysate has previously been studied on isolated hepatocyte cryopreservation, the authors using it directly again in the cryopreservation solution. It showed some interesting, though limited, results (Tolosa, L. et. al., Transplantation 2011; 91(12):1340-6) on cell viability with a reduction in apoptosis but no improvement on function. No longer-term preconditioning of the cells prior to cryopreservation is known.

Most cryopreservation solutions contain DMSO as an essential cryoprotectant. However, DMSO is deleterious to cells when they are exposed to it for more than 30 min. The incubation of cells in the cryopreservation medium is therefore always limited to 30 min. When dealing with microbeads, though, that time might not have been long enough to allow the full penetration of trehalose inside the cells. Therefore, the current invention studied the effects of a preconditioning with trehalose for a day—in culture medium at 37° C.—prior to the microbead cryopreservation. Culture medium usually contains foetal calf serum, which is strictly prohibited for cell therapy product manufacture. To replace this and provide necessary oncotic pressure to the cells, the current invention uses human albumin solution or platelet lysate, the supplement normally used to culture MSCs.

Preconditioning of the cells in a medium before cryopreservation is carried out for at least 10 minutes, at least 30 minutes, at least one hour, at least 2 hours, at least 5 hours, at least 10 hours, at least 12 hours, at least 15 hours, at least 20 hours, at least 24 hours or for at least 48 hours.

Preconditioning is carried out at 37° C., but can be carried about at 20-40° C., 30-40° C., 35-38° C., 36-37° C.

Cryopreservation is carried out at −70° C. or less, −80° C. or less, −90° C. or less, −100° C. or less, −110° C. or less, −120° C. or less, −130° C. or less, −150° C. or less, −170° C. or less, −200° C. or less.

Thawing after cryopreservation can be carried out at room temperature, 20-30° C., 35-37° C., or 37° C.

### Methods

Microbead preconditioning: The microbeads from the HMB002 production were put in culture for 20 hr in William's E medium (WEM, Sigma Aldrich) containing 2 mM L-glutamine (Gibco), 10 mM HEPES (Gibco), 10 mg/L Insulin, 5.5 mg/L Transferrin, 670 ug/L Sodium Selenite (ITS-G GIBCO), 10⁷ M Dexamethasone (Sigma Aldrich), 100 U/mL Penicillin and 100 μg/mL Streptomycin (Gibco). The medium was then supplemented with human albumin solution (Zenalb® 20, Bio Products Laboratory Limited, UK), or Pooled Human Platelet Lysate (PL; Stemulate™, COOK Regentec, USA), with or without trehalose (Sigma-Aldrich, Gillingham). The ratio of beads to medium was 1:4. Statistics were done using GraphPad Prism one-way ANOVA and multiple comparisons. Controls, n=2; PL, n=6; PL+Treh, n=6; HAS, n=4; HAS+Treh n=1. ****p<0.0001.

Microbead cryopreservation: Ice-cold cryopreservation medium was prepared as previously (UW solution with 5% glucose and 10% DMSO) and the controlled-rate freezer (Planer KRYO 10) set up. For each group, the beads were then loaded into cryopreservation bags and frozen as described previously (Jitrarauch, S., et. al., as above) before being stored at −150° C.

Microbead analysis: For recovery, the cryobags were quickly thawed in a 37° C. water bath with gentle agitation. The microbeads were then washed in cold culture medium to eliminate the DMSO, before transfer to culture in warm complete WEM (supplemented with 10% foetal calf serum, GE Hyclone). The cell viability was assessed using the fluorescent dye FDA, and microscopy, as mentioned above. The cell synthetic functions were assessed by the measurement of human A1AT, by ELISA as described above.

### Results

HMB002 microbeads were cryopreserved directly after production or after 20 hr of preconditioning in media containing human albumin solution or platelet lysate, with or without trehalose. Whatever their treatment, the beads were then frozen in the same cryopreservation medium (UW+DMSO+Glucose). As a precaution, the effects of the preconditioning on the cell viability were analysed. The various preconditioning did not result in any obvious loss of cell viability when assessed by FDA staining and microscopy before the cryopreservation (data not shown).

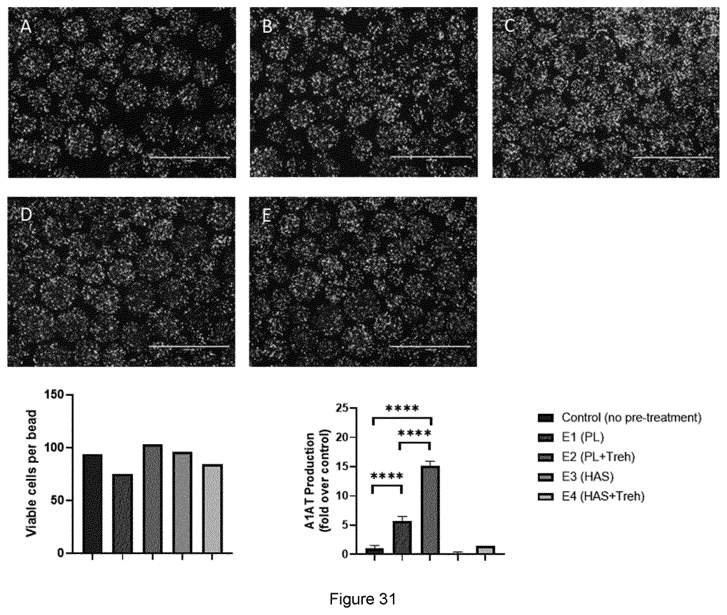

After thawing, the microbead viability was assessed again at day 0 and day 6 (see FIG. 31). The hepatocyte synthetic function (α-1 antitrypsin production—A1AT) was assessed over the first 24 hr after thawing. A minimum of 50 beads were analysed for the cell viability measurement. As compared to the control, the only group that seemed to display a lower cell viability, 6 days after thawing, was the one where the microbeads had been preconditioned with platelet lysate only (75 vs 94 live cells/bead). The preconditioning with human albumin did not seem to affect the cell viability (96 vs 94). The addition of trehalose to the platelet lysate preconditioning seemed to improve viability from 75 to 103 live cells per bead, though it did not have a similar effect when human albumin was used in the preconditioning medium (84 vs 96 live cells per beads for HAS+PL and HAS, respectively). Whilst the impact of the preconditioning had moderate effects on the cell viability, the consequences on cell function were striking. There was no positive effect of the use of preconditioning when human albumin was used in the culture medium: A1AT production was decreased to 20% of the control values—though the addition of trehalose seemed to improve this with a function raised to 142% (not reaching statistical significance in this setting). The use of platelet lysate in the preconditioning, though, had a very positive effect on the cell function, raising the A1AT production 5.6-fold. This had in turn a permissive action on the effect of trehalose, with a 15-fold improvement of A1AT secretion over the controls when the cells were preconditioned with both trehalose and platelet lysate.

1. A composition comprising a combination of hepatocytes and mesenchymal stromal cells (MSCs), wherein the cells are co-encapsulated within alginate to form microbeads, characterised in that the alginate is derivatised with a peptide comprising an Arg-Gly-Asp (RGD) motif.

2. The composition according to claim 1, wherein the peptide is represented by GRGDSP (SEQ ID NO: 1), optionally MVG GRGDSP.

3. The composition according to claim 1, wherein the alginate has a molecular weight in excess of 100,000.

4. The composition according to claim 1, any one of the preceding claims wherein the alginate comprises a G/M ratio of at least 1.5:1.

5. The composition according to claim 1, wherein the microbeads are prepared by crossing linking a solution of alginate in the presence of a divalent cation, or a combination thereof.

6. The composition according to claim 1, wherein the MSCs are derived from neonatal tissues, umbilical cords or placenta, or from adult tissue including bone marrow, adipose tissue and peripheral blood.

7. The composition according to claim 1, wherein the hepatocytes are hepatocyte-like cells and/or primary hepatocytes.

8. The composition according to claim 1, wherein the ratio of hepatocytes to MSCs is in the range of from 1:1 to 5:1, optionally wherein hepatocytes are present in excess.

9. The composition according to claim 1, further comprising a transplant, storage or cryopreservation media, optionally wherein the ratio of transplant medium to microbeads is about 1:1.

10. (canceled)

11. (canceled)

12. A method for preparing a composition according to claim 1, comprising:
a. admixing hepatocytes and MSCs with a solution of an alginate which has been derivatised with a peptide comprising an RGD motif:
b. cross-linking the alginate to produce microbeads in which the hepatocytes and MSCs are encapsulated; optionally
c. storing the microbeads in a suitable storage or cryopreservation medium until ready for use, or when ready for clinical use; and
d. adding the microbeads to a suitable transplant medium,
optionally wherein the cross linking is effected using a salt selected from an alkali or alkaline earth metal salt, or a combination thereof:
optionally wherein the cross linking is effected using a solution containing any divalent cations (e.g. Ca²⁺, Ba²⁺, Sr²⁺), optionally calcium chloride.

13. (canceled)

14. (canceled)

15. A method for the treatment or prophylaxis of liver disease, the method comprising administering to a patient in need thereof an effective amount of a composition according to claim 1.

16. A pharmaceutical composition comprising a therapeutically effective amount of the composition according to claim 1, optionally further comprising a pharmaceutically acceptable carrier, diluent or excipient, including combinations thereof.

17. (canceled)

18. (canceled)

19. The method according to claim 15, wherein the composition is administered to a subject in need as a single dose.

20. The method according to claim 15, wherein the dose administered is in the range of between about 5 ml to 30 ml microbeads per kg of body weight of the subject, in the range of between about 7 ml to 25 ml per kg of weight of the subject or in the range of between about 12 ml to 22 ml per kg of body weight of the subject.

21. (canceled)

22. (canceled)

23. The method according to claim 20, wherein the liver disease is acute liver failure (ALF), optionally wherein the subject-having ALF is a paediatric patient.

24. A kit comprising any one or more of: MSCs, hepatocytes, alginate derivatised with a peptide comprising an Arg-Gly-Asp (RGD) motif, and optionally a suitable transplant, storage or cryopreservation media, optionally further comprising directions for preparation and/or use of the components therein and/or tools suitable for the preparation and/or delivery of the microbeads to a subject.

25. (canceled)

26. The composition according to claim 1, wherein the composition is cryopreserved.

27. A method of cryopreservation of microbeads comprising the steps of
(a) preconditioning the microbeads in medium; and
(b) removing the microbead preconditioning medium and replacing it with a cryopreservation medium,
optionally wherein the microbeads cryopreserved comprise the microbeads of the composition of claim 1.

28. (canceled)

29. The method of claim 27, further comprising one or more of the following optional steps:
wherein the medium of the preconditioning step (a) comprises human albumin solution or platelet lysate,
wherein the medium of the preconditioning step (a) comprises trehalose,
optionally wherein optionally the preconditioning step (a) involves culturing the microbeads in medium for at least 10 minutes,
wherein the cryopreservation medium comprises University of Wisconsin solution (UW), DMSO and glucose.

30. (canceled)

31. (canceled)

32. (canceled)
